(19) United States
(12) Patent Application Publication
(10) Pub. No.: US 2012/0183660 A1
(43) Pub. Date: Jul. 19, 2012

(54) ISOMANNIDE DERIVATIVES AND THEIR USE AS TASTANTS

(75) Inventors: Catherine Tachdjian, San Diego, CA (US); Sara L. Adamski-Werner, San Diego, CA (US); Jeffrey M. Yamamoto, San Diego, CA (US); Hengyuan Lang, San Diego, CA (US); Donald S. Karanewsky, Escondido, CA (US)

(73) Assignee: Senomyx, Inc., San Diego, CA (US)

(21) Appl. No.: 13/381,458

(22) PCT Filed: Jun. 30, 2010
(86) PCT No.: PCT/US2010/040579

§ 371 Date: Mar. 23, 2012

Related U.S. Application Data
(60) Provisional application No. 61/222,659, filed on Jul. 2, 2009.

(51) Int. Cl. A23L 1/226; C07D 493/04

(52) U.S. Cl. 426/536; 549/464; 546/284.1

(57) ABSTRACT

The present invention provides isomannide derivatives having structural formula (I) as shown below and certain subgenera or species thereof, as flavoring agents or tastants, flavor or taste modifiers, and/or flavor/taste enhancers, particularly, savory (“umami”) flavoring agent or tastant, savory taste modifiers, and/or savory flavor enhancers, for ingestible compositions. The present invention also provides methods of preparing isomannide derivatives having the structural formula (II) shown below and certain subgenera or species thereof.

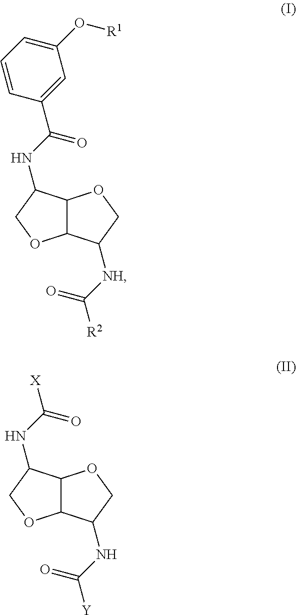

### CROSS-REFERENCE TO RELATED APPLICATION

The present application claims the benefit of U.S. Provisional Application No. 61/222,659, filed on Jul. 2, 2009 and entitled “ISOMANNIDE DERIVATIVES AND THEIR USE AS TASTANTS”, the content of which is incorporated by reference in its entirety for all purposes.

### FIELD OF THE INVENTION

The present invention relates to isomannide derivatives and their use as flavoring agents or tastants, flavor or taste modifiers, and/or flavor or taste enhancers for ingestible compositions.

### BACKGROUND OF THE INVENTION

For centuries, various natural and unnatural compositions and/or compounds have been added to foods, beverages, and/or comestible compositions to improve their taste. Although it has long been known that there are only a few basic types of “tastes” (sweet, sour, bitter, salty, and “umami”/savory), the biological and biochemical basis of taste perception was poorly understood, and most taste improving or taste modifying agents have been discovered largely by simple trial and error processes.

One of the five known basic tastes is the “savory” or “umami” flavor of monosodium glutamate (“MSG”), which is now commonly added to many food and beverage compositions to desirably improve their “savory” flavor. MSG is known to produce adverse reactions in some people, but very little progress has been made in identifying artificial substitutes for MSG. It is known that a few naturally occurring materials can increase or enhance the effectiveness of MSG as a savory flavoring agent, so that less MSG is needed for a given flavoring application. For example the naturally occurring nucleotide compounds inosine monophosphate (IMP) or guanosine monophosphate (GMP) are known to have a synergistic and/or multiplier effect on the savory taste of MSG. Nevertheless, IMP and GMP are both difficult and expensive to either synthesize or isolate and purify from natural sources and hence have limited practical application to many commercial needs in food compositions. Less expensive compounds that would provide and/or replace the flavor of MSG itself, or multiply the effectiveness of any MSG that is present, e.g. to replace the need for IMP or GMP additives, could be of very high value. This is especially true if the compounds could be used at low concentrations, so as to minimize costs and possible health risks.

In recent years substantial progress has been made in biotechnology and particularly in understanding the underlying biological and biochemical phenomena of taste perception. For example, taste receptor proteins involved in taste perception have been recently identified in mammals. In particular, two different families of G protein coupled receptors believed to be involved in taste perception, T2Rs and T1Rs, have been identified. (See, e.g., Nelson, et al., Cell (2001) 106(3):381-390; Adler, et al., Cell (2000) 100(6):693-702; Chandrashekar, et al., Cell (2000) 100:703-711; Matsunami, et al., Nature (2000) 404:601-604; Li, et al., Proc. Natl. Acad. Sci. USA (2002) 99:4962-4966; Montmayeur, et al., Nature Neuroscience (2001) 4(S):492-498: U.S. Pat. No. 6,462,148; and PCT publications WO 02/06254, WO 00/63166 art, WO 02/064631, and WO 03/001876, and U.S. Patent publication US 2003-0232407 A1). The entire disclosures of the articles, patent applications, and issued patents cited immediately above are hereby incorporated herein by reference, for all purposes, including a) their disclosures of the identities and structures of T2Rs and T1Rs mammalian taste receptor proteins; b) methods for artificially expressing those receptors in cell lines; c) using the resulting cell lines for screening compounds as potential “savory” flavoring agents; and d) assays and/or high throughput screens that measure T1R1/T1R3 or T1R2/T1R3 receptor activity by fluorometric imaging in the presence of the target compounds.

Whereas the T2R family includes over 25 genes that are involved in bitter taste perception, the T1R family includes only three members, T1R1, T1R2 and T1R3. (See Li, et al., Proc. Natl. Acad. Sci. USA (2002) 99:4962-4966.) Recently it was disclosed in WO 02/064631 and/or WO 03/001876 that certain T1R members, when co-expressed in suitable mammalian cell lines, assemble to form functional taste receptors. Particularly it was found that co-expression of T1R1 and T1R3 in a suitable host cell results in a functional T1R1/T1R3 savory (“umami”) taste receptor that responds to savory taste stimuli, including monosodium glutamate. (See Li, et al. (Id.).

Recently, certain U.S. and international patent applications have been filed by some of the current Applicants that disclosed the use of certain compounds as umami and/or sweet tastants, and/or synergistic enhancers of the “Umami” taste of MSG, and/or the sweet taste of a variety of natural and artificial sweeteners. See, for example, U.S. Provisional Patent Application Ser. No. 60/494,071 filed Aug. 6, 2003, U.S. Provisional Patent Application Ser. No. 60/552,064 filed Mar. 9, 2004, U.S. Utility patent application Ser. No. 10/913,303, filed Aug. 6, 2004 and published as U.S. Patent Publication Serial No. US-2005-0084506-A1 on Apr. 21, 2005; and PCT Patent Application Serial No. PCT/US04/25419 filed Aug. 6, 2004 and published as PCT Publication WO 2005/041684 on May 12, 2005, and PCT Publication WO 2005/015158 published on Feb. 17, 2005. The entire disclosures of the patent applications cited immediately above are hereby incorporated herein by this reference, for all purposes, including their disclosures of the identities and structures of compounds that can serve as potential “savory” or sweet flavoring agents or enhancers. Nevertheless, there is a continuing need for new and improved flavoring agents or tastants, flavor/taste modifiers, and flavor/taste enhancers.

### DETAILED DESCRIPTION OF THE INVENTION

The present invention can be understood more readily by reference to the following detailed description of various embodiments of the invention and the Examples included therein and to the chemical drawings and Tables and their previous and following description. Before the present compounds, compositions, and/or methods are disclosed and described, it is to be understood that unless otherwise specifically indicated by the claims, the invention is not limited to specific foods or food preparation methods, specific comestible carriers or formulations, or to particular modes of formulating the compounds of the invention into comestible products or compositions intended for oral administration, because as one of ordinary skill in relevant arts is well aware, such things can of course, vary. It is also to be understood that the terminology used herein is for the purpose of describing particular embodiments only and is not intended to be limiting.

### DEFINITIONS

A “comestibly acceptable carrier or excipient” is a solid or liquid medium and/or composition that is used to prepare a desired dispersed dosage form of the inventive compound, in order to administer the inventive compound in a dispersed/diluted form, so that the biological effectiveness of the inventive compound is maximized. Comestibly acceptable carriers includes many common food ingredients, such as water at neutral, acidic, or basic pH, fruit or vegetable juices, vinegar, marinades, beer, wine, natural water/fat emulsions such as milk or condensed milk, edible oils and shortenings, fatty acids and their alkyl esters, low molecular weight oligomers of propylene glycol, glyceryl esters of fatty acids, and dispersions or emulsions of such hydrophobic substances in aqueous media, salts such as sodium chloride, wheat flours, solvents such as ethanol, solid edible diluents such as vegetable powders or flours, or other liquid vehicles; dispersion or suspension aids; surface active agents; isotonic agents; thickening or emulsifying agents, preservatives; solid binders; lubricants and the like.

A “flavor” herein refers to the perception of taste and/or smell in a subject, which include sweet, sour, salty, bitter, umami, and others. The subject may be a human or an animal.

A “flavoring agent” herein refers to a compound or a biologically acceptable salt thereof that induces a flavor or taste in an animal or a human.

A “flavor modifier” herein refers to a compound or biologically acceptable salt thereof that modulates, including enhancing or potentiating, and inducing, the tastes and/or smell of a natural or synthetic flavoring agent in an animal or a human.

A “flavor enhancer” herein refers to a compound or biologically acceptable salt thereof that enhances and/or multiplies the tastes or smell of a natural or synthetic flavoring agent, or a comestible composition comprising the flavor enhancer.

“Savory flavor” herein refers to the savory “umami” taste typically induced by MSG (mono sodium glutamate) in an animal or a human.

“Savory flavoring agent,” “savory compound” or “savory receptor activating compound” herein refers to a compound or biologically acceptable salt thereof that elicits a detectable savory flavor in a subject, e.g., MSG (mono sodium glutamate) or a compound that activates a T1R1/T1R3 receptor in vitro. The subject may be a human or an animal. Compounds of the present invention are savory flavoring agents/compounds.

A “savory flavor modifier” herein refers to a compound or biologically acceptable salt thereof that modulates, including enhancing or potentiating, inducing, and blocking, the savory taste of a natural or synthetic savory flavoring agents, e.g., monosodium glutamate (MSG) in an animal or a human.

A “savory flavor enhancer” herein refers to a compound or biologically acceptable salt thereof that enhances or potentiates the savory taste of a natural or synthetic savory flavoring agents, e.g., monosodium glutamate (MSG) in an animal or a human.

An “umami receptor activating compound” herein refers to a compound that activates an umami receptor, such as a T1R1/T1R3 receptor.

An “umami receptor modulating compound” herein refers to a compound that modulates (activates, enhances or blocks) an umami receptor.

An “umami receptor enhancing compound” herein refers to a compound that enhances or potentiates the effect of a natural or synthetic umami receptor activating compound, e.g., monosodium glutamate (MSG).

A “savory flavor modulating amount” herein refers to an amount of a compound of Formula (I) that is sufficient to alter (either increase or decrease) savory taste in a comestible or medicinal product or composition, or a precursor thereof, sufficiently to be perceived by a human subject. In many embodiments of the invention, at least about 0.001 ppm of the heterocyclic compound would need to be present in order for most human subjects to perceive a modulation of the savory flavor of a comestible composition comprising the heterocyclic compound. A broad range of concentration that would typically be employed in order to economically provide a desirable degree of savory flavor modulation can be from about 0.001 ppm to 100 ppm, or a narrow range from about 0.1 ppm to about 10 ppm. Alternative ranges of savory flavor modulating amounts can be from about 0.01 ppm to about 30 ppm, from about 0.05 ppm to about 15 ppm, from about 0.1 ppm to about 5 ppm, or from about 0.1 ppm to about 3 ppm.

A “savory flavor enhancing amount” herein refers to an amount of a compound that is sufficient to enhance the taste of a natural or synthetic flavoring agents, e.g., monosodium glutamate (MSG) in a comestible or medicinal product or composition, as perceived by an animal or a human. A broad range of a savory flavor enhancing amount can be from about 0.001 ppm to 100 ppm, or a narrow range from about 0.1 ppm to about 10 ppm. Alternative ranges of savory flavor enhancing amounts can be from about 0.01 ppm to about 30 ppm, from about 0.05 ppm to about 15 ppm, from about 0.1 ppm to about 5 ppm, or from about 0.1 ppm to about 3 ppm.

An “umami receptor modulating amount” herein refers to an amount of a compound that is sufficient to modulate (activate, enhance or block) an umami taste receptor protein. In many embodiments of the invention, an umami receptor modulating amount is at least about 1 pM, or at least about 1 nM, or at least about 10 nM, or at least about 100 nM (i.e. about 0.1 μM). A “T1R1/T1R3 receptor modulating or activating amount” is an amount of compound that is sufficient to modulate or activate a T1R1/T1R3 receptor. These amounts are preferably the same as the umami receptor modulating amounts.

An “umami receptor” is a taste receptor that can be modulated by a savory compound. Preferably an umami receptor is a G protein coupled receptor, and more preferably the umami receptor is a T1R1/T1R3 receptor.

Compounds of the invention modulate an umami receptor and preferably are agonists of the T1R1/T1R3 receptor. An agonist of this receptor has the effect of activating a G protein signaling cascade. In many cases, this agonist effect of the compound on the receptor also produces a perceived savory flavor in a taste test. It is desirable, therefore, that such inventive compounds serve as a replacement or enhancer for MSG, which is not well tolerated by some in, for example, comestible products.

In addition, this agonist effect also is responsible for the synergistic savory taste effect, which occurs when a compound of the invention is combined with another savory flavoring agent such as MSG. The nucleotides, IMP or GMP, are conventionally added to MSG, to intensify the savory flavor of MSG, so that relatively less MSG is needed to provide the same savory flavor in comparison to MSG alone. Therefore, it is desirable that combining compounds of the invention with another savory flavoring agent such as MSG advantageously eliminates the need to add expensive nucleotides, such as IMP, as a flavor enhancer, while concomitantly reducing or eliminating the amount of a savory compound such as MSG needed to provide the same savory flavor in comparison to the savory compound or MSG alone.

A “synergistic effect” relates to the enhanced savory flavor of a combination of savory compounds or receptor activating compounds, in comparison to the sum of the taste effects or flavor associated effects associated with each individual compound. In the case of savory enhancer compounds, a synergistic effect on the effectiveness of MSG may be indicated for a compound of Formula (I) having an EC50 ratio (defined herein below) of 2.0 or more, or preferably 5.0 or more, or 10.0 or more, or 15.0 or more.

When the compounds described here include one or more chiral centers, the stereochemistry of such chiral centers can independently be in the R or S configuration, or a mixture of the two. The chiral centers can be further designated as R or S or R,S or d,D, l,L or d,l, D,L. Correspondingly, the compounds of the invention, if they can be present in optically active form, can be present in the form of a racemic mixture of enantiomers, or in the form of either of the separate enantiomers in substantially isolated and purified form, or as a mixture comprising any relative proportions of the enantiomers. Where so indicated in the claims herein, if a single enantiomer of the potentially optically active heterocyclic compounds disclosed is desired, for either health or efficacy reasons, preferably it is present in an enantiomeric excess of at least about 80%, or at least about 90%, or at least about 95%, or at least about 98%, or at least about 99%, or at least about 99.5%.

As used herein, “hydrocarbon residue” or “hydrocarbon radical” refers to a chemical sub-group or radical within a larger chemical compound which contains only carbon and hydrogen atoms. The hydrocarbon residue may be aliphatic or aromatic, straight-chain, cyclic, branched, saturated or unsaturated. In many embodiments the hydrocarbon residues are of limited dimensional size and molecular weight, and may comprise 1 to 18 carbon atoms, 1 to 16 carbon atoms, 1 to 12 carbon atoms, 1 to 10 carbon atoms, 1 to 8 carbon atoms, 1 to 6 carbon atoms, or 1 to 4 carbon atoms.

The hydrocarbon residue, when described as “substituted,” contains or is substituted with one or more independently selected heteroatoms such as O, S, N, P, or the halogens (fluorine, chlorine, bromine, and iodine), or one or more substituent groups containing heteroatoms (OH, NH₂, NO₂, SO₃H, and the like) over and above the carbon and hydrogen atoms of the substituent residue. Substituted hydrocarbon residues may also contain carbonyl groups, amino groups, hydroxyl groups and the like, or contain heteroatoms inserted into the “backbone” of the hydrocarbon residue.

As used herein, “inorganic” group or residue refers to a neutral, cationic, or anionic radical substituents on the organic molecules disclosed or claimed herein that have from one to 16 atoms that do not include carbon, but do contain other heteroatoms from the periodic table that preferably include one or more atoms independently selected from the group consisting of H, O, N, S, one or more halogens, or alkali metal or alkaline earth metal ions. Examples of inorganic radicals include, but are not limited to, H, Na+, Ca++ and K+, halogens which include fluorine, chlorine, bromine, and iodine, OH, SH, SO₃H, SO₃⁻, PO₃H, PO₃⁻, NO, NO² or NH₂, and the like.

As used herein, the term “alkyl,” “alkenyl” and “alkynyl” include straight- and branched-chain and cyclic monovalent substituents that respectively are saturated, unsaturated with at least one double bond, and unsaturated with at least one triple bond.

“Alkyl” refers to a hydrocarbon group that can be conceptually formed from an alkane by removing hydrogen from the structure of a non-cyclic hydrocarbon compound having straight or branched carbon chains, and replacing the hydrogen atom with another atom or organic or inorganic substituent group. In some embodiments of the invention, the alkyl groups are “C1 to C6 alkyl” such as methyl, ethyl, propyl, isopropyl, n-butyl, iso-butyl, sec-butyl, tert-butyl, amyl, tert-amyl, hexyl and the like. Many embodiments of the invention comprise “C1 to C4 alkyl” groups (alternatively termed “lower alkyl” groups) that include, but are not limited to, methyl, ethyl, propyl, iso-propyl n-butyl, iso-butyl, sec-butyl, and t-butyl groups. Some of the preferred alkyl groups of the invention have three or more carbon atoms preferably 3 to 16 carbon atoms, 4 to 14 carbon atoms, or 6 to 12 carbon atoms. The term “alkenyl” is structurally analogous to an alkyl group or residue that comprises at least one carbon-carbon double bond. In some embodiments, alkenyl groups are “C2 to C7 alkenyls” which are exemplified by vinyl, allyl, 2-butenyl, 3-butenyl, 2-pentenyl, 3-pentenyl, 4-pentenyl, 2-hexenyl, 3-hexenyl, 4-hexenyl, 5-hexenyl, 2-heptenyl, 3-heptenyl, 4-heptenyl, 5-heptenyl, 6-heptenyl, as well as dienes and trienes of straight and branched chains. In other embodiments, alkenyls are limited to two to four carbon atoms.

The term “alkynyl” is analogous to an alkyl group or radical that comprises at least one carbon-carbon triple bond. Preferred alkynyl groups are “C2 to C7 alkynyl” such as ethynyl, propynyl, 2-butynyl, 2-pentynyl, 3-pentynyl, 2-hexynyl, 3-hexynyl, 4-hexynyl, 2-heptynyl, 3-heptynyl, 4-heptynyl, 5-heptynyl as well as di- and tri-ynes of straight and branched chains including ene-ynes.

The terms “substituted alkyl,” “substituted alkenyl,” “substituted alkynyl,” and “substituted alkylene” denote that the alkyl, alkenyl, or alkynyl groups or radicals as described herein, wherein one or more hydrogen atoms has been conceptually substituted by one or more, and preferably one or two independently selected organic or inorganic substituent groups or radicals, that can include a halogen, hydroxy, amino, SH, a C1 to C7 alkoxy, or alkoxy-alkyl, oxo, C3 to C7 cycloalkyl, naphthyl, amino, (monosubstituted)amino, (disubstituted)amino, guanidino, heterocycle, substituted heterocycle, imidazolyl, indolyl, pyrrolidinyl, C1 to C7 acyl, C1 to C7 acyloxy, nitro, carboxy, carbamoyl, carboxamide, N—(C1 to C6 alkyl)carboxamide, N,N-di(C1 to C6 alkyl)carboxamide, cyano, methylsulfonylamino, thiol, C1 to C4 alkylthio or C1 to C4 alkylsulfonyl groups. The substituted alkyl groups may be substituted once or more, and preferably once or twice, with the same or with different substituents. In many embodiments of the invention, a preferred group of substituent groups for a substantial alkyls include hydroxy, fluoro, chloro, NH₂, NHCH₃, N(CH₃)₂, CO₂CH₃, SEt, SCH₃, methyl, ethyl, isopropyl, vinyl, trifluoromethyl, methoxy, ethoxy, isopropoxy, and trifluoromethoxy groups. In many embodiments of the invention that comprise the above lists of substituent groups, an even more preferred group of substituent groups include hydroxy, SEt, SCH₃, methyl, ethyl, isopropyl, trifluoromethyl, methoxy, ethoxy, and trifluoromethoxy groups.

Examples of the above substituted alkyl groups include the 2-oxo-prop-1-yl, 3-oxo-but-1-yl, cyanomethyl, nitromethyl, chloromethyl, trifluoromethyl, hydroxymethyl, tetrahydropyranyloxymethyl, trityloxymethyl, propionyloxymethyl, aminomethyl, carboxymethyl, allyloxycarbonylmethyl, allyloxycarbonylaminomethyl, methoxymethyl, ethoxymethyl, t-butoxymethyl, acetoxymethyl, chloromethyl, trifluoromethyl, 6-hydroxyhexyl, 2,4-dichloro(n-butyl), 2-aminopropyl, 1-chloroethyl, 2-chloroethyl, 1-bromoethyl, 2-chloroethyl, 1-fluoroethyl, 2-fluoroethyl, 1-iodoethyl, 2-iodoethyl, 1-chloropropyl, 2-chloropropyl, 3-chloropropyl, 1-bromopropyl, 2-bromopropyl, 3-bromopropyl, 1-fluoropropyl, 2-fluoropropyl, 3-fluoropropyl, 2-aminoethyl, 1-aminoethyl, N-benzoyl-2-aminoethyl, N-acetyl-2-aminoethyl, N-benzoyl-1-aminoethyl, N-acetyl-1-aminoethyl and the like.

Examples of substituted alkenyl groups include styrenyl, 3-chloro-propen-1-yl, 3-chloro-buten-1-yl, 3-methoxy-propen-2-yl, 3-phenyl-buten-2-yl, 1-cyano-buten-3-yl and the like. The geometrical isomerism is not critical, and all geometrical isomers for a given substituted double bond can be included.

Examples of substituted alkynyl groups include phenylacetylen-1-yl, 1-phenyl-2-propyn-1-yl and the like.

“Heteroalkyl” refers to alkyl group in which one or more of the carbon atoms (and optionally any associated hydrogen atoms), are each, independently of one another, replaced with the same or different heteroatoms or heteroatomic groups. Typical heteroatoms or heteroatomic groups which can replace the carbon atoms include, but are not limited to, O, S, N, Si, —NH—, —S(O)—, —S(O)₂—, —S(O)NH—, —S(O)₂NH— and the like and combinations thereof. The heteroatoms or heteroatomic groups may be placed at any interior position of the alkyl, alkenyl or alkynyl groups. Typical heteroatomic groups which can be included in these groups include, but are not limited to, —O—, —S—, —O—O—, —S—S—, —O—S—, —NR₂—, ═N—N═, —N═N—, —N═N—NR₂, —PR—, —P(O)₂—, —POR—, —O—P(O)₂—, —SO—, —SO₂—, —SnR₂— and the like, where R is each independently hydrogen, alkyl, substituted alkyl, aryl, substituted aryl, arylalkyl, substituted arylalkyl, cycloalkyl, substituted cycloalkyl, cycloheteroalkyl, substituted cycloheteroalkyl, heteroalkyl, substituted heteroalkyl, heteroaryl, substituted heteroaryl, heteroarylalkyl or substituted heteroarylalkyl.

“Haloalkyl” is substituted alkyl group or residue wherein one or more hydrogens of the corresponding alkyl group have been replaced with a halogen atom (fluorine, chlorine, bromine, and iodine). Preferred haloalkyls can have one to four carbon atoms. Examples of preferred haloalkyl groups include trifluoromethyl and pentafluoroethyl groups.

Haloalkoxy groups are alkoxy groups or residues wherein one or more hydrogens from the R group of the alkoxy group are a halogen atom (fluorine, chlorine, bromine, and iodine). Preferred haloalkoxy groups can have one to four carbon atoms. Examples of preferred haloalkoxy groups include trifluoromethyoxy and pentafluoroethoxy groups.

The term “oxo” denotes a carbon atom bonded to two additional carbon atoms substituted with an oxygen atom doubly bonded to the carbon atom, thereby forming a ketone radical or residue.

“Alkoxy” or “alkoxyl” refers to an —OR radical or group, wherein R is an alkyl radical. In some embodiments the alkoxy groups can be C1 to C8, and in other embodiments can be C1 to C4 alkoxy groups wherein R is a lower alkyl, such as a methoxy, ethoxy, n-propoxy, isopropoxy, n-butoxy, t-butoxy and like alkoxy groups. The term “substituted alkoxy” means that the R group is a substituted alkyl group or residue. Examples of substituted alkoxy groups include trifluoromethoxy, hydroxymethyl, hydroxyethyl, hydroxypropyl, and alkoxyalkyl groups such as methoxymethyl, methoxyethyl, polyoxoethylene, polyoxopropylene, and similar groups.

“Alkylamine” refers to —NHR or —NR₂, wherein each R is independently an alkyl radical. In some embodiments the alkylamine groups can be C1 to C8, and in other embodiments can be C1 to C4 alkylamine groups wherein R is a lower alkyl, such as a methylamine, ethylamine, propylamine, N,N-methylethylamine, N,N-dimethylamine, N,N-diethylamine, N,N-diisopropylamine and like alkylamine groups.

“Alkoxyalkyl” refers to an —R—O—R′ group or radical, wherein R and R′ are alkyl groups. In some embodiments the alkoxyalkyl groups can be C1 to C8, and in other embodiments can be C1 to C4. In many embodiments, both R and R′ are a lower alkyl, such as a methoxy, ethoxy, n-propoxy, isopropoxy, n-butoxy, t-butoxy and like alkoxy groups. Examples of alkoxyalkyl groups include, methoxymethyl, ethoxyethyl, methoxypropyl, and methoxybutyl and similar groups.

“Acyloxy” refers to an RCO₂— ester group where R is an alkyl, cycloalkyl, aryl, heteroaryl, substituted alkyl, substituted cycloalkyl, substituted aryl, or substituted heteraryl group or radical wherein the R radical comprises one to seven or one to four carbon atoms. In many embodiments, R is an alkyl radical, and such acyloxy radicals are exemplified by formyloxy, acetoxy, propionyloxy, butyryloxy, pivaloyloxy, pentanoyloxy, hexanoyloxy, heptanoyloxy and the like. In other embodiments the R groups are C1-C4 alkyls.

As used herein, “acyl” encompasses the definitions of alkyl, alkenyl, alkynyl and the related hetero-forms which are coupled to an additional organic residue through a carbonyl group to form a ketone radical or group. Preferred acyl groups are “C1 to C7 acyl” such as formyl, acetyl, propionyl, butyryl, pentanoyl, pivaloyl, hexanoyl, heptanoyl, benzoyl and the like. More preferred acyl groups are acetyl and benzoyl.

The term “substituted acyl” denotes an acyl group wherein the R group substituted by one or more, and preferably one or two, halogen, hydroxy, oxo, alkyl, cycloalkyl, naphthyl, amino, (monosubstituted)amino, (disubstituted)amino, guanidino, heterocyclic ring, substituted heterocyclic ring, imidazolyl, indolyl, pyrrolidinyl, C1 to C7 alkoxy, alkoxy-alkyl, C1 to C7 acyl, C1 to C7 acyloxy, nitro, C1 to C6 alkyl ester, carboxy, alkoxycarbonyl, carbamoyl, carboxamide, N—(C1 to C6 alkyl)carboxamide, N,N-di(C1 to C6 alkyl)carboxamide, cyano, methylsulfonylamino, thiol, C1 to C4 alkylthio or C1 to C4 alkylsulfonyl groups. The substituted acyl groups may be substituted once or more, and preferably once or twice, with the same or with different substituents.

Examples of C1 to C7 substituted acyl groups include 4-phenylbutyroyl, 3-phenylbutyroyl, 3-phenylpropanoyl, 2-cyclohexanylacetyl, cyclohexanecarbonyl, 2-furanoyl and 3 dimethylaminobenzoyl.

“Carbocyclyl”, as used herein, denotes a saturated, unsaturated, or partially saturated hydrocarbon radical having some or all carbon atoms linked in one or more ring structures. Carbocyclyl includes cycloalkyl, cycloalkylene, cycloalkenyl, cycloalkenylene, and the like, such as, for example, cyclohexylmethylene.

Cycloalkyl residues or groups are structurally related to cyclic monocyclic or bicyclic hydrocarbon compounds wherein one or more hydrogen atoms has been replaced with an organic or inorganic substituent group. The cycloalkyls of the current inventions comprise at least 3 up to 12, or more preferably 3 to 8 ring carbon atoms, or more preferably 4 to 6 ring carbon atoms. Examples of such cycloalkyl residues include cyclopropyl, cyclobutyl, cyclopentyl, cyclohexyl, cycloheptyl, cyclooctyl rings, and saturated bicyclic or fused polycyclic cycloalkanes such as decalin groups, polycyclic norbornyl or adamantly groups, and the like.

Preferred cycloalkyl groups include “C3 to C7 cycloalkyl” such as cyclopropyl, cyclobutyl, cyclopentyl, cyclohexyl or cycloheptyl rings. Similarly, the term “C5 to C7 cycloalkyl” includes cyclopentyl, cyclohexyl or cycloheptyl rings.

“Substituted cycloalkyl” denote a cycloalkyl rings as defined above, substituted by 1 to four, or preferably one or two substituents independently selected from a halogen, hydroxy, C1 to C4 alkylthio, C1 to C4 alkylsulfoxide, C1 to C4 alkylsulfonyl, C1 to C4 substituted alkylthio, C1 to C4 substituted alkylsulfoxide, C1 to C4 substituted alkylsulfonyl, C1 to C4 alkyl, C1 to C4 alkoxy, C1 to C6 substituted alkyl, C1 to C4 alkoxy-alkyl, oxo (monosubstituted)amino, (disubstituted)amino, trifluoromethyl, carboxy, phenyl, substituted phenyl, phenylthio, phenylsulfoxide, phenylsulfonyl, amino. In many embodiments of substituted cycloalkyl groups, the substituted cycloalkyl group will have 1, 2, 3, or 4 substituent groups independently selected from hydroxy, fluoro, chloro, NH₂, NHCH₃, N(CH₃)₂, CO₂CH₃, SEt, SCH₃, methyl, ethyl, isopropyl, vinyl, trifluoromethyl, methoxy, ethoxy, isopropoxy, and trifluoromethoxy groups.

The term “cycloalkylene” means a cycloalkyl, as defined above, where the cycloalkyl radical is bonded at two positions connecting together two separate additional groups. Similarly, the term “substituted cycloalkylene” means a cycloalkylene where the cycloalkyl radical is bonded at two positions connecting together two separate additional groups and further bearing at least one additional substituent.

The term “cycloalkenyl” indicates preferably a 1,2, or 3-cyclopentenyl ring, a 1,2,3 or 4-cyclohexenyl ring or a 1,2,3,4 or 5-cycloheptenyl ring, while the term “substituted cycloalkenyl” denotes the above cycloalkenyl rings substituted with a substituent, preferably by a C1 to C6 alkyl, halogen, hydroxy, C1 to C7 alkoxy, alkoxy-alkyl, trifluoromethyl, carboxy, alkoxycarbonyl oxo, (monosubstituted)amino, (disubstituted)amino, phenyl, substituted phenyl, amino, or protected amino.

The term “cycloalkenylene” is a cycloalkenyl ring, as defined above, where the cycloalkenyl radical is bonded at two positions connecting together two separate additional groups. Similarly, the term “substituted cycloalkenylene” means a cycloalkenylene further substituted preferably by halogen, hydroxy, C1 to C4 alkylthio, C1 to C4 alkylsulfoxide, C1 to C4 alkylsulfonyl, C1 to C4 substituted alkylthio, C1 to C4 substituted alkylsulfoxide, C1 to C4 substituted alkylsulfonyl, C1 to C6 alkyl, C1 to C7 alkoxy, C1 to C6 substituted alkyl, C1 to C7 alkoxy-alkyl, oxo, (monosubstituted)amino, (disubstituted)amino, trifluoromethyl, carboxy, alkoxycarbonyl, phenyl, substituted phenyl, phenylthio, phenylsulfoxide, phenylsulfonyl, amino, or substituted amino group.

The term “heterocyclyl” or “heterocyclic ring” denotes optionally substituted 3 to 8-membered rings having one or more carbon atoms connected in a ring that also comprise 1 to 5 ring heteroatoms, such as oxygen, sulfur and/or nitrogen inserted into the ring. These heterocyclic rings can be saturated, unsaturated or partially unsaturated, but are preferably saturated. Preferred unsaturated heterocyclic rings include furanyl, thiofuranyl, pyrrolyl, pyridyl, pyrimidyl, pyrazinyl, benzoxazole, benzthiazole, quinolinlyl, and like heteroaromatic rings. Preferred saturated heterocyclic rings include piperidyl, aziridinyl, piperidinyl, piperazinyl, tetrahydrofurano, pyrrolyl, and tetrahydrothiophenyl rings.

The term “substituted heterocycle” or “substituted heterocyclic ring” means the above-described heterocyclic ring is substituted with, for example, one or more, and preferably one or two, substituents which are the same or different which substituents preferably can be halogen, hydroxy, thio, alkylthio, cyano, nitro, C1 to C4 alkyl, C1 to C4 alkoxy, C1 to C4 substituted alkoxy, alkoxy-alkyl, C1 to C4 acyl, C1 to C4 acyloxy, carboxy, alkoxycarbonyl, carboxymethyl, hydroxymethyl, alkoxy-alkyl amino, monosubstituted)amino, (disubstituted)amino carboxamide, N—(C1 to C6 alkyl)carboxamide, N,N-di(C1 to C6 alkyl)carboxamide, trifluoromethyl, N—((C1 to C6 alkyl)sulfonyl)amino, N-(phenylsulfonyl)amino groups, or substituted with a fused ring, such as benzo-ring. In many embodiments of substituted heterocyclic groups, the substituted cycloalkyl group will have 1, 2, 3, or 4 substituent groups independently selected from hydroxy, fluoro, chloro, NH₂, NHCH₃, N(CH₃)₂, CO₂CH₃, SEt, SCH₃, methyl, ethyl, isopropyl, vinyl, trifluoromethyl, methoxy, ethoxy, isopropoxy, and trifluoromethoxy groups.

An “aryl” group refers to a monocyclic, linked bicyclic or fused bicyclic radical or group comprising at least one six membered aromatic “benzene” ring. Aryl groups preferably comprise between 6 and 12 ring carbon atoms, and are exemplified by phenyl, biphenyl, naphthyl, indanyl, and tetrahydronapthyl groups. Aryl groups can be optionally substituted with various organic and/or inorganic substituent groups, wherein the substituted aryl group in combination with all its substituents comprise between 6 and 18, or preferably 6 and 16 total carbon atoms. Preferred optional substituent groups include 1, 2, 3, or 4 substituent groups independently selected from hydroxy, fluoro, chloro, NH₂, NHCH₃, N(CH₃)₂, CO₂CH₃, SEt, SCH₃, methyl, ethyl, isopropyl, vinyl, trifluoromethyl, methoxy, ethoxy, isopropoxy, and trifluoromethoxy groups.

The term “heteroaryl” means a heterocyclic aryl derivative which preferably contains a five-membered or six-membered conjugated and aromatic ring system having from 1 to 4 heteroatoms independently selected from oxygen, sulfur and/or nitrogen, inserted into the unsaturated and conjugated heterocyclic ring. Heteroaryl groups include monocyclic heteroaromatic, linked bicyclic heteroaromatic or fused bicyclic heteroaromatic moieties. Examples of heteroaryls include pyridinyl, pyrimidinyl, and pyrazinyl, pyridazinyl, pyrrolyl, furanyl, thiofuranyl, oxazoloyl, isoxazolyl, phthalimido, thiazolyl, quinolinyl, isoquinolinyl, indolyl, or a furan or thiofuran directly bonded to a phenyl, pyridyl, or pyrrolyl ring and like unsaturated and conjugated heteroaromatic rings. Any monocyclic, linked bicyclic, or fused bicyclic heteroaryl ring system which has the characteristics of aromaticity in terms of electron distribution throughout the ring system is included in this definition. Typically, the heteroaromatic ring systems contain 3-12 ring carbon atoms and 1 to 5 ring heteroatoms independently selected from oxygen, nitrogen, and sulfur atoms.

The term “substituted heteroaryl” means the above-described heteroaryl is substituted with, for example, one or more, and preferably one or two, substituents which are the same or different which substituents preferably can be halogen, hydroxy, protected hydroxy, thio, alkylthio, cyano, nitro, C1 to C6 alkyl, C1 to C7 substituted alkyl, C1 to C7 alkoxy, C1 to C7 substituted alkoxy, alkoxy-alkyl, C1 to C7 acyl, C1 to C7 substituted acyl, C1 to C7 acyloxy, carboxy, alkoxycarbonyl, carboxymethyl, hydroxymethyl, amino, (monosubstituted)amino, (disubstituted)amino, carboxamide, N—(C1 to C6 alkyl)carboxamide, N,N-di(C1 to C6 alkyl)carboxamide, trifluoromethyl, N—((C1 to C6 alkyl)sulfonyl)amino or N-(phenylsulfonyl)amino groups. In many embodiments of substituted heteroaryl groups, the substituted cycloalkyl group will have 1, 2, 3, or 4 substituent groups independently selected from hydroxy, fluoro, chloro, NH₂, NHCH₃, N(CH₃)₂, CO₂CH₃, SEt, SCH₃, methyl, ethyl, isopropyl, vinyl, trifluoromethyl, methoxy, ethoxy, isopropoxy, and trifluoromethoxy groups.

Similarly, “arylalkyl” and “heteroarylalkyl” refer to aromatic and heteroaromatic systems which are coupled to another residue through a carbon chain, including substituted or unsubstituted, saturated or unsaturated, carbon chains, typically of 1-6C. These carbon chains may also include a carbonyl group, thus making them able to provide substituents as an acyl moiety. Preferably, arylalkyl or heteroarylalkyl is an alkyl group substituted at any position by an aryl group, substituted aryl, heteroaryl or substituted heteroaryl. Preferred groups also include benzyl, 2-phenylethyl, 3-phenyl-propyl, 4-phenyl-n-butyl, 3-phenyl-n-amyl, 3-phenyl-2-butyl, 2-pyridinylmethyl, 2-(2-pyridinyl)ethyl, and the like.

The term “substituted arylalkyl” denotes an arylalkyl group substituted on the alkyl portion with one or more, and preferably one or two, groups preferably chosen from halogen, hydroxy, oxo, amino, (monosubstituted)amino, (disubstituted)amino, guanidino, heterocyclic ring, substituted heterocyclic ring, C1 to C6 alkyl, C1 to C6 substituted alkyl, C1 to C7 alkoxy, C1 to C7 substituted alkoxy, alkoxy-alkyl, C1 to C7 acyl, C1 to C7 substituted acyl, C1 to C7 acyloxy, nitro, carboxy, alkoxycarbonyl, carbamoyl, carboxamide, N—(C1 to C6 alkyl)-carboxamide, N,N—(C1 to C6 dialkyl)carboxamide, cyano, N—(C1 to C6 alkylsulfonyl)amino, thiol, C1 to C4 alkylthio, C1 to C4 alkylsulfonyl groups; and/or the phenyl group may be substituted with one or more, and preferably one or two, substituents preferably chosen from halogen, hydroxy, protected hydroxy, thio, alkylthio, cyano, nitro, C1 to C6 alkyl, C1 to C6 substituted alkyl, C1 to C7 alkoxy, C1 to C7 substituted alkoxy, alkoxy-alkyl, C1 to C7 acyl, C1 to C7 substituted acyl, C1 to C7 acyloxy, carboxy, alkoxycarbonyl, carboxymethyl, hydroxymethyl, amino, (monosubstituted)amino, (disubstituted)amino, carboxamide, N—(C1 to C6 alkyl) carboxamide, N,N-di(C1 to C6 alkyl)carboxamide, trifluoromethyl, N—((C1 to C6 alkyl)sulfonyl)amino, N-(phenylsulfonyl)amino, cyclic C2 to C7 alkylene or a phenyl group, substituted or unsubstituted, for a resulting biphenyl group. The substituted alkyl or phenyl groups may be substituted with one or more, and preferably one or two, substituents which can be the same or different.

Examples of the term “substituted arylalkyl” include groups such as 2-phenyl-1-chloroethyl, 2-(4-methoxyphenyl)ethyl, 4-(2,6-dihydroxy phenyl)-n-hexyl, 2-(5-cyano-3-methoxyphenyl)-n-pentyl, 3-(2,6-dimethylphenyl)propyl, 4-chloro-3-aminobenzyl, 6-(4-methoxyphenyl)-3-carboxy-n-hexyl, 5-(4-aminomethylphenyl)-3-(aminomethyl)-n-pentyl, 5-phenyl-3-oxo-n-pent-1-yl and the like.

The term “arylalkylene” specifies an arylalkyl, as defined above, where the arylalkyl radical is bonded at two positions connecting together two separate additional groups. The definition includes groups of the formula: -phenyl-alkyl- and alkyl-phenyl-alkyl-. Substitutions on the phenyl ring can be 1,2, 1,3 or 1,4. The term “substituted arylalkylene” is an arylalkylene as defined above that is further substituted preferably by halogen, hydroxy, protected hydroxy, C1 to C4 alkylthio, C1 to C4 alkylsulfoxide, C1 to C4 alkylsulfonyl, C1 to C4 substituted alkylthio, C1 to C4 substituted alkylsulfoxide, C1 to C4 substituted alkylsulfonyl, C1 to C6 alkyl, C1 to C7 alkoxy, C1 to C6 substituted alkyl, C1 to C7 alkoxy-alkyl, oxo, (monosubstituted)amino, (disubstituted)amino, trifluoromethyl, carboxy, alkoxycarbonyl, phenyl, substituted phenyl, phenylthio, phenylsulfoxide, phenylsulfonyl, amino, or protected amino group on the phenyl ring or on the alkyl group.

The term “substituted phenyl” specifies a phenyl group substituted with one or more, and preferably one or two, moieties preferably chosen from the groups consisting of halogen, hydroxy, protected hydroxy, thio, alkylthio, cyano, nitro, C1 to C6 alkyl, C1 to C6 substituted alkyl, C1 to C7 alkoxy, C1 to C7 substituted alkoxy, alkoxy-alkyl, C1 to C7 acyl, C1 to C7 substituted acyl, C1 to C7 acyloxy, carboxy, alkoxycarbonyl, carboxymethyl, hydroxymethyl, amino, (monosubstituted)amino, (disubstituted)amino, carboxamide, N—(C1 to C6 alkyl)carboxamide, N,N-di(C1 to C6 alkyl)carboxamide, trifluoromethyl, N—((C1 to C6 alkyl)sulfonyl)amino, N-(phenylsulfonyl)amino or phenyl, wherein the phenyl is substituted or unsubstituted, such that, for example, a biphenyl results. In many embodiments of substituted phenyl groups, the substituted cycloalkyl group will have 1, 2, 3, or 4 substituent groups independently selected from hydroxy, fluoro, chloro, NH₂, NHCH₃, N(CH₃)₂, CO₂CH₃, SEt, SCH₃, methyl, ethyl, isopropyl, vinyl, trifluoromethyl, methoxy, ethoxy, isopropoxy, and trifluoromethoxy groups.

The terms “halo” and “halogen” refer to fluoro, chloro, bromo or iodo atoms or ions. Preferred halogens are chloro and fluoro. Although many of the compounds of the invention having halogen atoms as substituents are highly effective in binding to the relevant Umami taste receptors, such halogenated organic compounds can in some cases have undesirable toxicological properties when administered to an animal in vivo. Therefore, in the various embodiments of the compounds of Formula (I), if a halogen atom (including a fluoro, chloro, bromo, or iodo atom) is listed as a possible substituent, an alternative and preferred group of substituents expressly contemplated hereby would not include the halogen groups.

The term “(monosubstituted)amino” refers to an amino (NHR) group wherein the R group is chosen from the group consisting of phenyl, C6-C10 substituted phenyl, C1 to C6 alkyl, C1 to C6 substituted alkyl, C1 to C7 acyl, C1 to C7 substituted acyl, C2 to C7 alkenyl, C2 to C7 substituted alkenyl, C2 to C7 alkynyl, C2 to C7 substituted alkynyl, C7 to C12 phenylalkyl, C7 to C12 substituted phenylalkyl and heterocyclic ring. The (monosubstituted)amino can additionally have an amino-protecting group as encompassed by the term “protected (monosubstituted)amino.”

The term “(disubstituted)amino” refers to an amino group (NR2) with two substituents independently chosen from the group consisting of phenyl, C6-C10 substituted phenyl, C1 to C6 alkyl, C1 to C6 substituted alkyl, C1 to C7 acyl, C2 to C7 alkenyl, C2 to C7 alkynyl, C7 to C12 phenylalkyl, and C7 to C12 substituted phenylalkyl. The two substituents can be the same or different.

The term “alkylthio” refers to —SR groups wherein R is an optionally substituted C1-C7 or C1-C4 organic group, preferably an alkyl, cycloalkyl, aryl, or heterocyclic group, such as methylthio, ethylthio, n-propylthio, isopropylthio, n-butylthio, t-butylthio and like groups.

The term “alkylsulfoxide” indicates —SO₂R groups wherein R is an optionally substituted C1-C7 or C1-C4 organic group, preferably an alkyl, cycloalkyl, aryl, or heterocyclic group, such as methylthio, ethylthio, n-propylthio, isopropylthio, n-butylthio, t-butylthio and like groups, such as methylsulfoxide, ethylsulfoxide, n-propylsulfoxide, isopropylsulfoxide, n-butylsulfoxide, sec-butylsulfoxide and the like.

The term “alkylsulfonyl” indicates —S(O)R groups wherein R is an optionally substituted C1-C7 or C1-C4 organic group, which include for example groups such as methylsulfonyl, ethylsulfonyl, n-propylsulfonyl, isopropylsulfonyl, n-butylsulfonyl, t-butylsulfonyl and the like.

The terms “phenylthio,” “phenylsulfoxide,” and “phenylsulfonyl” specify a sulfoxide (—S(O)—R), or sulfone (—SO₂R) wherein the R group is a phenyl group. The terms “substituted phenylthio,” “substituted phenylsulfoxide,” and “substituted phenylsulfonyl” means that the phenyl of these groups can be substituted as described above in relation to “substituted phenyl.”

The term “alkoxycarbonyl” means an “alkoxy” group attached to a carbonyl group, (i.e. a carboxylic acid ester, —C(O)—OR, wherein R is preferably an alkyl group, preferably a C1-C4 alkyl group. The term “substituted alkoxycarbonyl” denotes a substituted alkoxy bonded to the carbonyl group, which alkoxy may be substituted as described above in relation to substituted alkyl.

The term “phenylene” means a phenyl group where the phenyl radical is bonded at two positions connecting together two separate additional groups. Examples of “phenylene” includes 1,2-phenylene, 1,3-phenylene, and 1,4-phenylene.

The term “substituted alkylene” means an alkyl group where the alkyl radical is bonded at two positions connecting together two separate additional groups and further bearing an additional substituent. Examples of “substituted alkylene” includes aminomethylene, 1-(amino)-1,2-ethyl, 2-(amino)-1,2-ethyl, 1-(acetamido)-1,2-ethyl, 2-(acetamido)-1,2-ethyl, 2-hydroxy-1,1-ethyl, 1-(amino)-1,3-propyl.

“Substituted,” when used to modify a specified group or radical, means that one or more hydrogen atoms of the specified group or radical are each, independently of one another, replaced with the same or different substituent(s). Substituent groups useful for substituting saturated carbon atoms in the specified group or radical include, but are not limited to —R^{a}, halo, —O⁻, ═O, —OR^{b}, —SR^{b}, —S⁻, ═S, —NR^{c}R^{c}, ═NR^{b}, ═N—OR^{b}, trihalomethyl, —CF₃, —CN, —OCN, —SCN, —NO, —NO₂, ═N₂, —N₃, —S(O)₂R^{b}, —S(O)₂NR^{b}, —S(O)₂O⁻, —S(O)₂OR^{b}, —OS(O)₂R^{b}, —OS(O)₂O⁻, —OS(O)₂OR^{b}, —P(O)(O⁻)₂, —P(O)(OR^{b})(O⁻), —P(O)(OR^{b})(OR^{b}), —C(O)R^{b}, —C(S)R^{b}, —C(NR^{b})R^{b}, —C(O)O⁻, —C(O)OR^{b}, —C(S)OR^{b}, —C(O)NR^{c}R^{c}, —C(NR^{b})NR^{c}R^{c}, —OC(O)R^{b}, —OC(S)R^{b}, —OC(O)O⁻, —OC(O)OR^{b}, —OC(S)OR^{b}, —NR^{b}C(O)R^{b}, —NR^{b}C(S)R^{b}, —NR^{b}C(O)O⁻, —NR^{b}C(O)OR^{b}, —NR^{b}C(S)OR^{b}, —NR^{b}C(O)NR^{c}R^{c}, —NR^{b}C(NR^{b})R^{b} and —NR^{b}C(NR^{b})NR^{c}R^{c}, where R^{a} is selected from the group consisting of alkyl, cycloalkyl, heteroalkyl, cycloheteroalkyl, aryl, arylalkyl, heteroaryl and heteroarylalkyl; each R^{b} is independently hydrogen or R^{a}; and each R^{c} is independently R^{b} or alternatively, the two R^{c}s may be taken together with the nitrogen atom to which they are bonded form a 4-, 5-, 6- or 7-membered cycloheteroalkyl which may optionally include from 1 to 4 of the same or different additional heteroatoms selected from the group consisting of O, N and S. As specific examples, —NR^{c}R^{c} is meant to include —NH₂, —NH-alkyl, N-pyrrolidinyl and N-morpholinyl. As another specific example, a substituted alkyl is meant to include -alkylene-O-alkyl, -alkylene-heteroaryl, -alkylene-cycloheteroalkyl, -alkylene-C(O)OR^{b}, -alkylene-C(O)NR^{b}R^{b}, and —CH₂—CH₂—C(O)—CH₃. The one or more substituent groups, taken together with the atoms to which they are bonded, may form a cyclic ring including cycloalkyl and cycloheteroalkyl.

One or more of the compounds of the invention, may be present as a salt. The term “salt” encompasses those salts that form with the carboxylate anions and amine nitrogens and include salts formed with the organic and inorganic anions and cations discussed below. Furthermore, the term includes salts that form by standard acid-base reactions with basic groups (such as nitrogen containing heterocycles or amino groups) and organic or inorganic acids. Such acids include hydrochloric, hydrofluoric, trifluoroacetic, sulfuric, phosphoric, acetic, succinic, citric, lactic, maleic, fumaric, palmitic, cholic, pamoic, mucic, D-glutamic, D-camphoric, glutaric, phthalic, tartaric, lauric, stearic, salicyclic, methanesulfonic, benzenesulfonic, sorbic, picric, benzoic, cinnamic, and like acids.

By “Solvate”, it is meant a complex formed by solvation (the combination of solvent molecules with molecules or ions of the present compounds), or an aggregate that consists of a solute ion or molecule of the present compounds with one or more solvent molecules. When water is the solvent, the corresponding solvate is “hydrate”.

The term “N-oxide”, also known as amine-N-oxide or amine oxide, is a chemical compound that contains the functional group R₃N⁺—O⁻ (sometimes written as R₃N═O or R₃N→O).

The term “organic or inorganic cation” refers to positively charged counter-ions for the carboxylate anion of a carboxylate salt. Inorganic positively charged counter-ions include but are not limited to the alkali and alkaline earth metals, (such as lithium, sodium, potassium, calcium, magnesium, etc.) and other divalent and trivalent metallic cations such as barium, aluminum and the like, and ammonium (NH4)⁺ cations. Organic cations include ammonium cations derived from acid treatment or alkylation of primary-, secondary, or tertiary amines such as trimethylamine, cyclohexylamine; and the organic cations, such as dibenzylammonium, benzylammonium, 2-hydroxyethylammonium, bis(2-hydroxyethyl)ammonium, phenylethylbenzylammonium, dibenzylethylenediammonium, and like cations. See, for example, “Pharmaceutical Salts,” Berge, et al., J. Pharm. Sci. (1977) 66:1-19, which is incorporated herein by reference. Other cations encompassed by the above term include the protonated form of procaine, quinine and N-methylglucosamine, and the protonated forms of basic amino acids such as glycine, ornithine, histidine, phenylglycine, lysine and arginine. Furthermore, any zwitterionic form of the instant compounds formed by a carboxylic acid and an amino group is referred to by this term. For example, a cation for a carboxylate anion will exist when R² or R³ is substituted with a (quaternary ammonium)methyl group. A preferred cation for the carboxylate anion is the sodium cation.

The compounds of the invention can also exist as solvates and hydrates. Thus, these compounds may crystallize with, for example, waters of hydration, or one, a number of, or any fraction thereof of molecules of the mother liquor solvent. The solvates and hydrates of such compounds are included within the scope of this invention.

The term “amino acid” includes any one of the twenty naturally-occurring amino acids or the D-form of any one of the naturally-occurring amino acids. In addition, the term “amino acid” also includes other non-naturally occurring amino acids besides the D-amino acids, which are functional equivalents of the naturally-occurring amino acids. Such non-naturally-occurring amino acids include, for example, norleucine (“Nle”), norvaline (“Nva”), L- or D-naphthalanine, ornithine (“Orn”), homoarginine (homoArg) and others well known in the peptide art, such as those described in M. Bodanzsky, “Principles of Peptide Synthesis,” 1st and 2nd revised ed., Springer-Verlag, New York, N.Y., 1984 and 1993, and Stewart and Young, “Solid Phase Peptide Synthesis,” 2nd ed., Pierce Chemical Co., Rockford, Ill., 1984, both of which are incorporated herein by reference. Amino acids and amino acid analogs can be purchased commercially (Sigma Chemical Co.; Advanced Chemtech) or synthesized using methods known in the art.

A residue or radical of a chemical species, as used in the specification and concluding claims, refers to a structural fragment, or a moiety that is the resulting product of the chemical species in a particular reaction scheme or subsequent formulation or chemical product, regardless of whether the structural fragment or moiety is actually obtained from the chemical species. Thus, an ethylene glycol residue in a polyester refers to one or more —OCH₂CH₂O— repeat units in the polyester, regardless of whether ethylene glycol is used to prepare the polyester.

The term “organic residue” or “organic radical” defines a carbon containing residue or radical, comprising at least one carbon atom. Organic residues can contain one or more heteroatoms, or be bonded to another molecule through a heteroatom, including oxygen, nitrogen, sulfur, phosphorus, or the like. Examples of organic residues include but are not limited alkyl or substituted alkyls, alkoxyls or substituted alkoxyls, hydroxyalkyls and alkoxyalkyls, cycloalkyl or substituted cycloalkyls, cycloalkenyl or substituted cycloalkyenyls, heterocycles and substituted heterocycles, aryls and substituted aryls, heteroaryls and substituted heteroaryls, mono or di-substituted amino, amide groups, CN, CO₂H, CHO, COR′, CO₂R′, SR′ wherein R′ is an alkyl, and the like. Examples of species of organic groups or residues include but are not limited to NH₂, NHCH₃, N(CH₃)₂, CO₂CH₃, SEt, SCH₃, methyl, ethyl, isopropyl, vinyl, trifluoromethyl, methoxy, ethoxy, isopropoxy, trifluoromethoxy, phenyl, phenoxyl, and pyridyl groups or residues, and the like. Organic residues can comprise 1 to 18 carbon atoms, 1 to 15, carbon atoms, 1 to 12 carbon atoms, 1 to 8 carbon atoms, or 1 to 4 carbon atoms.

By the term “effective amount” of a compound as provided herein is meant a sufficient amount of the compound to provide the desired regulation of a desired function, such as protein function, or the induction of a particular type of taste perception. As will be pointed out below, the exact amount required will vary from subject to subject, depending on the species, age, general condition of the subject, specific identity and formulation of the comestible composition, etc. Thus, it is not possible to specify an exact “effective amount.” However, an appropriate effective amount can be determined by one of ordinary skill in the art using only routine experimentation.

The term “therapeutically effective amount” means the amount of a compound as provided herein that, when administered to a patient for treating a disease, is sufficient to effect such treatment for the disease. The “therapeutically effective amount” will vary depending on the compound, the disease and its severity and the age, weight, etc., of the patient to be treated. When the present compound is in a pharmaceutical composition, it can be in a therapeutically effective amount, or alternatively, not in a therapeutically effective amount. In other words, when the present compound is in a pharmaceutical composition, it can be either an active ingredient or inactive ingredient.

“Vehicle” refers to a diluent, adjuvant, excipient or carrier with which a compound is administered.

“Activating reagent”, as used herein, denotes a reagent which can react with one of the starting materials of a chemical reaction to form one or more active intermediate which subsequently facilitates the completion of the reaction. The active intermediate may not be stable enough to be separated and characterized. Examples of the activating reagent include, but are not limited to the coupling reagents used in amide/peptide synthesis, such as carbodiimide compound (EDC, DCC, DIC, and the like) and benzotriazole compounds (such as HOBt and HOAt); certain oxides and chloride (such as P₂O₅ and POCl₃); a reagent which react with a molecule to form a leaving group (such as MsCl, Tf₂O, and reagents for Mitsunobu reaction); and etc.

By “leaving group”, it is meant a functional group capable of detaching from a chemical substance. Examples of leaving group include, but are not limited to alkoxy, hydroxyl, carboxylate, fluoro, chloro, bromo, iodo, azide, thiocyanate, nitro, mesylate (—OMs), tosylate (—OTs), triflate (—OTf), and etc.

It must be noted that, as used in the specification and the appended claims, the singular forms “a,” “an” and “the” include plural referents unless the context clearly dictates otherwise. Thus, for example, reference to “an aromatic compound” includes mixtures of aromatic compounds. Furthermore, “a compound of the present invention”, “the present compound”, or “the compound of the present invention” refers to any compound and compounds covered by the generic formula (I) and/or any subgenus thereof including racemic mixtures, diastereomers, enantiomers, tautomers, or any other isomers and analogs (such as N-oxide) thereof as well as salts, solvates (such as, e.g., hydrate), esters, and/or prodrugs thereof.

Often, ranges are expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

“Optional” or “optionally” means that the subsequently described event or circumstance may or may not occur, and that the description includes instances where said event or circumstance occurs and instances where it does not. For example, the phrase “optionally substituted lower alkyl” means that the lower alkyl group may or may not be substituted and that the description includes both unsubstituted lower alkyl and lower alkyls where there is substitution.

Compounds of the Present Invention and Process for Preparing them

In one embodiment, the present invention provides compounds having structural formula (I):

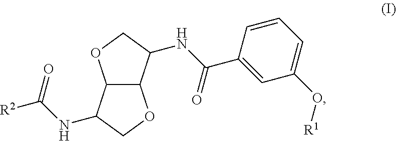

or the salt, solvate, ester, or N-oxide thereof.

In one embodiment of formula (I), R¹ is alkyl, substituted alkyl, alkenyl, substituted alkenyl, aryl, substituted aryl, carbocyclyl, or substituted carbocyclyl.

In one embodiment of formula (I), R² is alkyl, substituted alkyl, alkenyl, substituted alkenyl, aryl, substituted aryl, carbocyclyl, substituted carbocyclyl, heteroaryl, or substituted heteroaryl.

In one embodiment of formula (I), when R² is cyclopropyl; then R¹ is not phenyl, tolyl, methoxyphenyl, or pyridyl.

In one embodiment of formula (I), R¹ is alkyl, substituted alkyl, cycloalkyl, substituted cycloalkyl, phenyl, substituted phenyl; and R² is alkyl, substituted alkyl, cycloalkyl, substituted cycloalkyl, phenyl, substituted phenyl, pyridyl, or substituted pyridyl.

In one embodiment of formula (I), the compound has a structural formula (Ia):

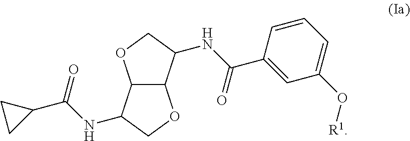

In one embodiment of formula (Ia), R¹ is alkyl, substituted alkyl, cycloalkyl, substituted cycloalkyl, or substituted phenyl.

In one embodiment of formula (I), the compound has a structural formula (Ib):

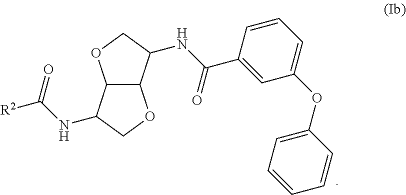

In one embodiment of formula (Ib), R² is alkyl, substituted alkyl, cycloalkyl, substituted cycloalkyl, phenyl, substituted phenyl, pyridyl, or substituted pyridyl.

In some specific embodiments of formula (I), the compounds have structural formulas selected from the group consisting of

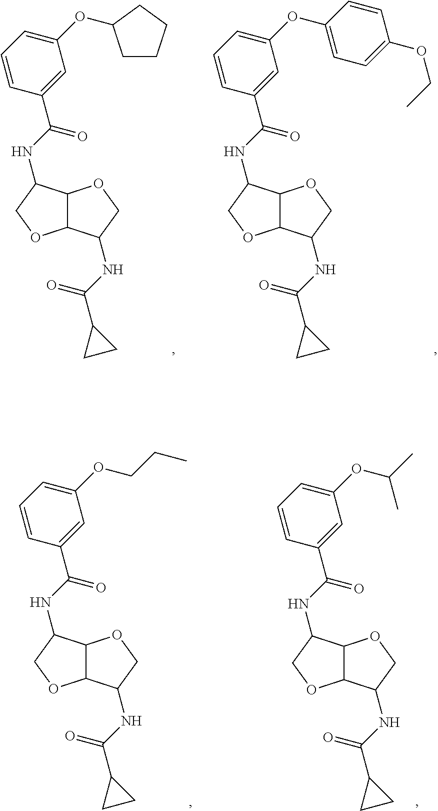

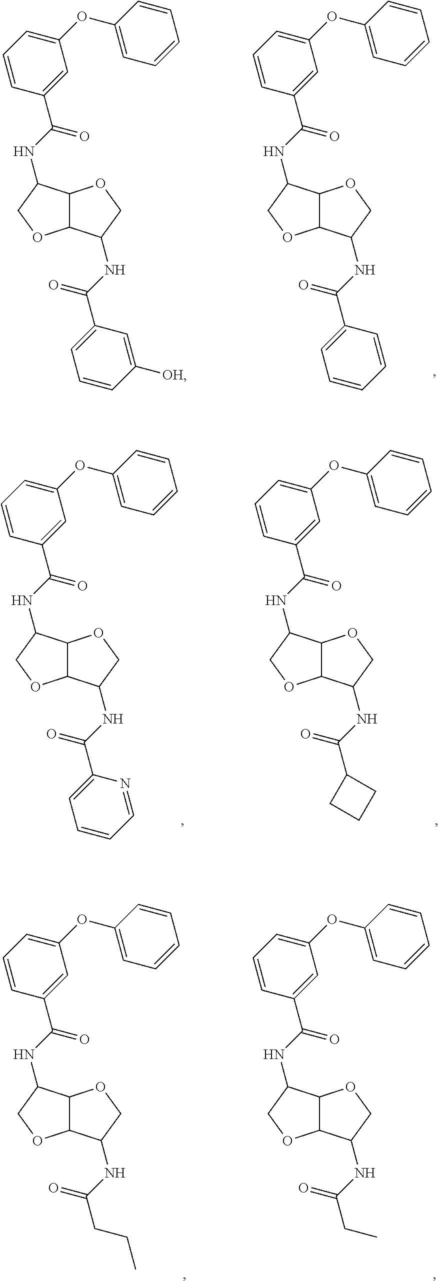

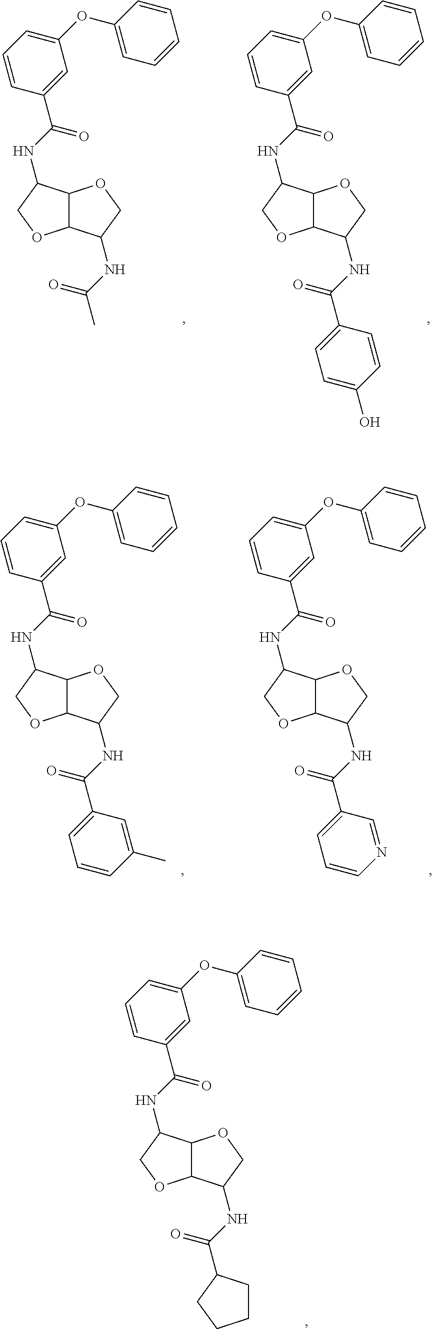

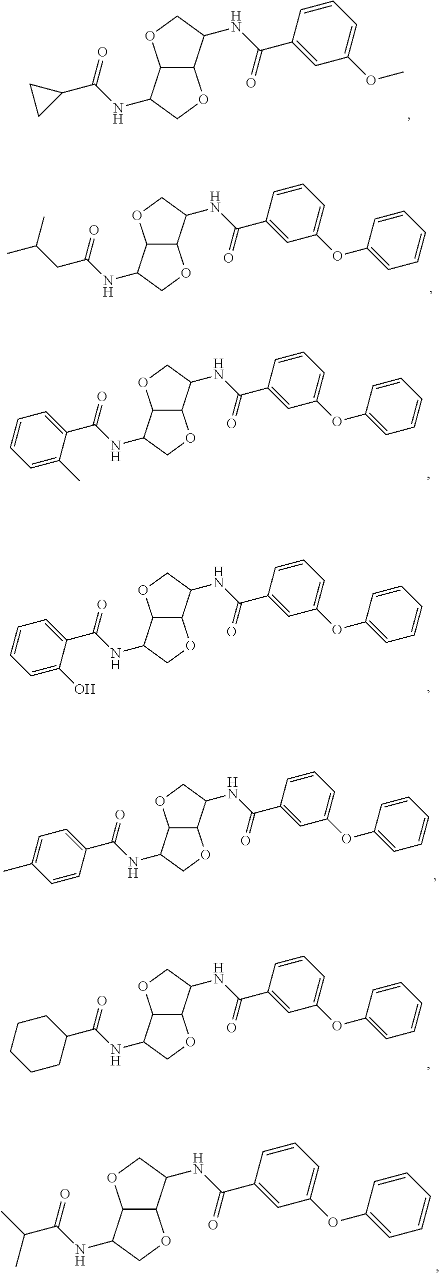

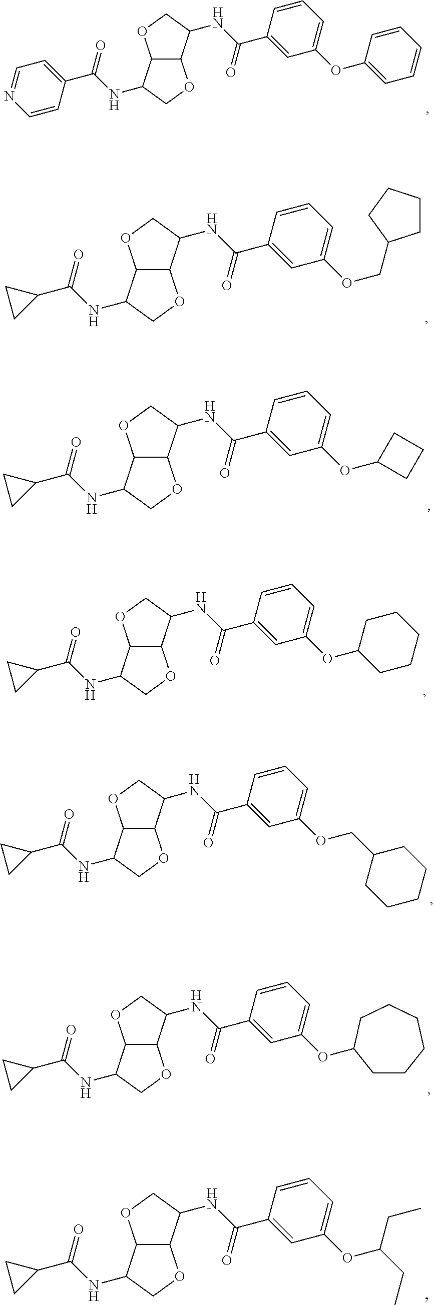

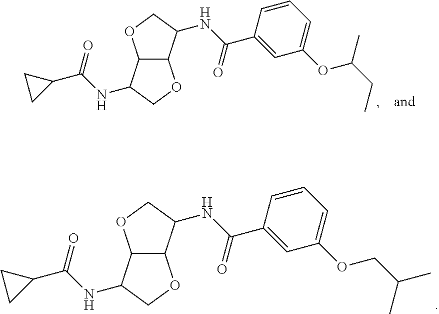

The compounds of the present invention can be prepared via synthetic methods known to one skilled in the art. For example, the present compounds can be synthesized according to the methods and procedures described in U.S. patent application Ser. No. 12/396,917, filed Mar. 3, 2009 and entitled “Isosorbide Derivatives and Their Use as Flavor Modifiers, Tastants, and Taste Enhancers”, the content of which is hereby incorporated by reference in its entirety. Alternatively, the present compounds can be synthesized according to the methods and procedures described hereinbelow.

In one embodiment, the present invention provides a method of preparing a compound of formula (II) in a large scale, such as an industrial scale,

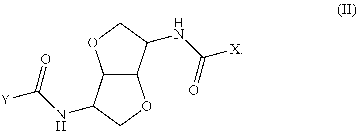

The present method utilizes commercially available building blocks and avoids applying chemical reactions with potential safety concerns, such as the azide and/or triflate formation. Thus, the present method can be safely and economically scaled up to an industrial production setting. In one embodiment, the present method uses reactions including leaving group formation, displacement with benzylamine, reductive hydrogenation including catalytic hydrogenation, and amide formation. In one embodiment, the present method includes preparation of a compound of formula (II) in a solid form substantially without chromatographic purification. By “substantially without chromatographic purification”, it is meant that chromatographic purification is not used to purify the crude material or is used occasionally as a secondary means to improve reaction yield or product purity. In the present method, chromatographic purification is used minimally or is not used at all.

In one embodiment, the present method comprises the steps of

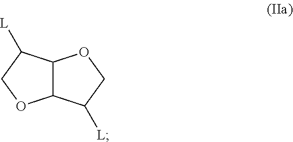

converting isomannide to a compound of formula (IIa):

reacting the compound of formula (IIa) with phenylmethanamine to obtain a compound of formula (IIb):

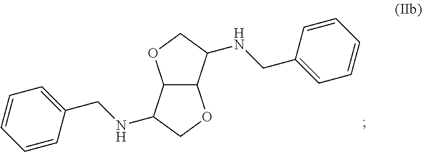

reducing the compound of formula (IIb) to a compound of formula (IIc):

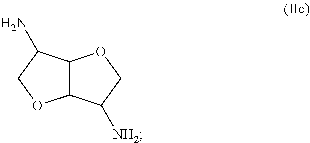

and

reacting the compound of formula (IIc) sequentially with a compound of formula (IId):

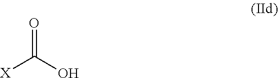

and a compound of formula (IIe):

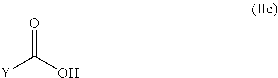

in the presence of one or more activating reagent. The desired product in each of the above-described steps can be obtained as a solid via trituration, crystallization, and/or other methods known to one skilled in the art. Chromatographic purification, such as silica column, may be applied occasionally to the residue of each of the above-described reaction to improve the reaction yield.

The conversion step can be carried out by any leaving group formation reaction. For example, the leaving group can be formed by reacting isomannide with an appropriate halid in the presence of a base. The base can be an organic or inorganic base. In one embodiment of the method, L is a leaving group. In one embodiment, L is halo, —OMs, —OTs, or —OTf.

In one embodiment of the method, X and Y are each independently alkyl, substituted alkyl, alkenyl, substituted alkenyl, alkynyl, substituted alkylnyl, alkoxy, alkylamine, heteroalkyl, substituted heteroalkyl, aryl, substituted aryl, arylalkyl, substituted arylalkyl, arylalkenyl, substituted arylalkenyl, heteroaryl, substituted heteroaryl, heterocyclyl, substituted heterocyclyl, heteroarylalkyl, substituted heteroarylalkyl, heterocyclylalkyl, substituted heterocyclylalkyl, carbocyclyl, or substituted carbocyclyl. In another embodiment, X and Y are each independently selected from alkyl, substituted alkyl, alkenyl, substituted alkenyl, alkylamine, aryl, substituted aryl, heteroaryl, substituted heteroaryl, arylalkyl, substituted arylalkyl, heterocyclyl, substituted heterocyclyl, heteroarylalkyl, substituted heteroarylalkyl, carbocyclyl, or substituted carbocyclyl.

The reduction step can be carried out by any chemical reaction that reduces a benzylamino group to an amino group, such as a catalytic hydrogenation. The suitable catalysts include, but are not limited to, precious metal-based catalysts and non-precious metal-based catalysts. Examples of the metal include platinum, palladium, rhodium, nickel, rhodium iridium, and combinations thereof. Moreover, the catalysts may be homogenous or heterogenous (such as a metal modified by an organic ligand or inorganic salt). The catalytic hydrogenation can be conducted at the atmospheric pressure or higher pressure. In one embodiment, the catalytic hydrogenation is conducted in a sealed vessel under pressurized condition to speed up the reaction process.

In one embodiment of the method, the one or more activating reagent is selected from a group consisting of a carbodiimide, a benzotrizaole compound, a phosphorus oxide or chloride, and a combination thereof.

One specific embodiment is described in Scheme A below:

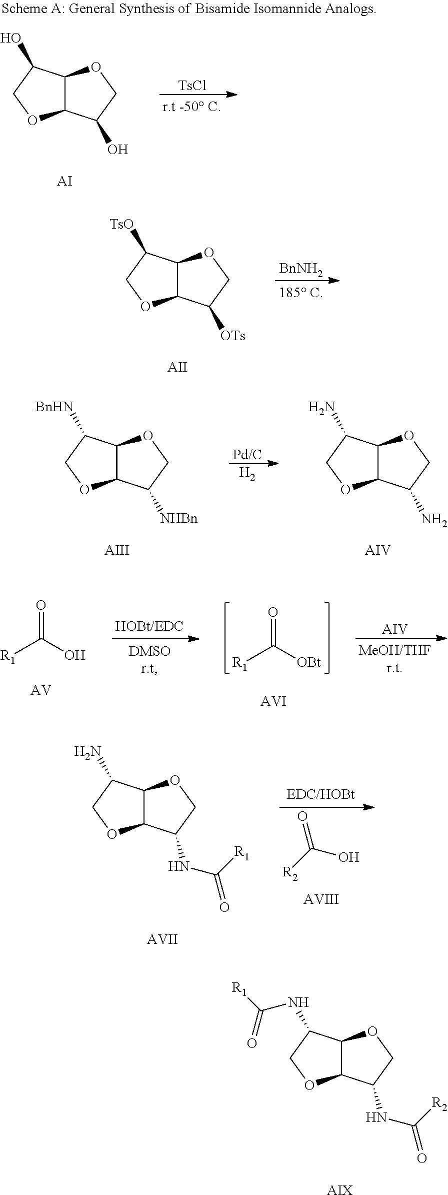

Isomannide analogs can be synthesized on a large scale starting from isomannide AI. Isonamide AI can be reacted with para-toluene sulfonyl chloride to provide the di-tosylate AII that is reacted with benzylamine to provides the diamine AIII with the desired stereochemistry. Hydrogenolysis under atmospheric pressure at room temperature affords the diamine AIV. Reaction of an appropriate carboxylic acid AV with 1-Hydroxy benzotriazole monohydrate and 1-(3-Dimethylaminopropyl)-3-ethylcarbodiimide hydrochloride in DMSO affords the HOBt ester intermediate AVI, which is then reacted with the diamine AIV to provide the monosubstituted amide AVII. An additional amide coupling with an appropriate carboxylic acid AVIII provides the desired, differentially substituted isomannide bisamide analogs AIX.

### Using the Compounds of Formula (I) or (II) to Prepare Compositions

The compounds of Formula (I) or (II) and its various compound sub-genuses and species, as described above are intended to be savory flavorant compounds or flavor modifiers for comestible products. As is apparent from the teachings and Examples herein, many compounds of Formula (I) or (II) are agonists of an hT1R1/hT1R3 “savory” receptor, at least at concentrations of about 100 μM or less. Accordingly many of the compounds of Formula (I) or (II) have a significant savory flavor independent of the presence or absence of MSG, and therefore can have utility as independent savory flavorants or flavor enhancers.

Nevertheless, it is preferable to use as little of such artificial flavorants as possible, so as to minimize both cost and undesirable health side effects of administration of the compounds of Formula (I) or (II) at high concentration levels. Accordingly, it is desirable to test the compounds of Formula (I) or (II) for their effectiveness as taste receptor agonists at lower concentration levels, so as to identify the best and most effective compounds of Formula (I) or (II). As was disclosed in WO 03/001876, and U.S. Patent Publication US 2003/0232407 A1, hereby incorporated herein by reference and as described hereinbelow, laboratory procedures now exist for measuring the agonist activities of compounds for an hT1R1/hT1R3 “savory” receptor. Such measurement methods typically measure an “EC₅₀”, i.e. the concentration at which the compound causes 50% activation of the relevant receptor.

Preferably, the compounds of Formula (I) or (II) that are savory flavor modifiers have an EC₅₀ for the hT1R1/hT1R3 receptor of less than about 10 μM. More preferably, such compounds have an EC₅₀ for the hT1R1/hT1R3 receptor of less than about 5 μM, 3 μM, 2 μM, 1 μM, or 0.5 μM.

In some embodiments, the compounds of Formula (I) or (II) are savory flavor modulators or enhancers of the agonist activity of monosodium glutamate for an hT1R1/hT1R3 receptor. Hereinbelow is described an assay procedure for so-called EC₅₀ ratios, i.e. for dissolving a compound of Formula (I) or (II) in water containing MSG, and measuring the degree to which the compound lowers the amount of MSG required to activate 50% of the available hT1R1/hT1R3 receptors. Preferably, the compounds of Formula (I) or (II), when dissolved in an aqueous solution comprising about 1 μM of the compound will decrease the observed EC₅₀ of monosodium glutamate for an hT1R1/hT1R3 receptor expressed in an HEK293-Gα15 cell line by at least 50%, i.e. the compound will have an EC₅₀ ratio of at least 2.0, or preferably 3.0, 5.0, or 7.0.

The above identified assays are useful in identifying the most potent of the compounds of Formula (I) or (II) for savory taste modifier or enhancer properties, and the results of such assays are believed to correlate well with actual savory taste perception in animals and humans, but ultimately the results of the assays can be confirmed, at least for the most potent of the compounds of Formula (I) or (II), by human taste testing. Such human taste testing experiments can be well quantified and controlled by tasting the candidate compounds in aqueous solutions, as compared to control aqueous solution, or alternatively by tasting the compounds of the inventions in actual food compositions.

Accordingly, in order to identify the more potent of the savory taste modifiers or agents, a water solution comprising a savory flavor modifying amount of any particular compound of Formula (I) or (II) or one of its subgenuses should have a savory taste as judged by the majority of a panel of at least eight human taste testers.

Correspondingly, in order to identify the more potent of the savory taste enhancers, a water solution, comprising a savory flavor modifying amount of a compound of Formula (I) or (II) and 12 mM monosodium glutamate, would have an increased savory taste as compared to a control water solution comprising 12 mM monosodium glutamate, as determined by the majority of a panel of at least eight human taste testers. Preferably, in order to identify the more potent of the savory taste enhancers, a water solution comprising a savory flavor modifying amount (preferably about 30, 10, 5, or 2 ppm) of the compound of Formula (I) or (II) and 12 mM monosodium glutamate will have an increased savory taste as compared to a control water solution comprising 12 mM monosodium glutamate and 100 μM inosine monophosphate, as determined by the majority of a panel of at least eight human taste testers.

### Using the Compounds of Formula (I) or (II) to Prepare Ingestible Compositions

Flavoring agents, flavor modifiers, and/or flavor enhancers including savory (“umami”) flavoring agents, savory flavor modifiers, and/or flavor enhancers, prepared from the compounds of Formula (I) or (II) and its various subgenera and species compounds herein, and their commestibly acceptable salts, and compositions thereof, have application in foods, beverages and other comestible compositions wherein savory compounds, especially MSG, IMP, or GMP are conventionally utilized. These compositions include compositions for human and animal consumption. This includes food or drinks (liquids) for consumption by agricultural animals, pets and zoo animals.

As used herein, the terms “ingestible composition” and “comestible composition” are used exchangeably and refer to any composition that can be taken by mouth. The ingestible or comestible composition may be taken by the mouth for any purpose including digestion, chewing, cleaning, etc. The ingestible composition includes both “food/beverage” and “non-edible products”. For example, the ingestible composition includes food/beverage compositions or products and medicinal compositions or products. “Food” herein means any edible product intended for consumption by humans or animals, including solids, semi-solids, or liquids (e.g., beverages). The term “food” and the term “food and beverage” are herein used interchangeably.

Those of ordinary skill in the art of preparing and selling comestible compositions (i.e edible foods or beverages, or precursors or flavor modifiers thereof) are well aware of a large variety of classes, subclasses and species of the comestible compositions, and utilize well-known and recognized terms of art to refer to those comestible compositions while endeavoring to prepare and sell various of those comestible compositions. Such a list of terms of art is enumerated below, and it is specifically contemplated hereby that the various subgenuses and species of the compounds of Formula (I) or (II) could be used to modify or enhance the savory flavors of the following list comestible compositions, either singly or in all reasonable combinations or mixtures thereof: one or more confectioneries, chocolate confectionery, tablets, countlines, bagged selflines/softlines, boxed assortments, standard boxed assortments, twist wrapped miniatures, seasonal chocolate, chocolate with toys, alfajores, other chocolate confectionery, mints, standard mints, power mints, boiled sweets, pastilles, gums, jellies and chews, toffees, caramels and nougat, medicated confectionery, lollipops, liquorice, other sugar confectionery, gum, chewing gum, sugarised gum, sugar-free gum, functional gum, bubble gum, bread, packaged/industrial bread, unpackaged/artisanal bread, pastries, cakes, packaged/industrial cakes, unpackaged/artisanal cakes, cookies, chocolate coated biscuits, sandwich biscuits, filled biscuits, savoury biscuits and crackers, bread substitutes, breakfast cereals, rte cereals, family breakfast cereals, flakes, muesli, other rte cereals, children's breakfast cereals, hot cereals, ice cream, impulse ice cream, single portion dairy ice cream, single portion water ice cream, multi-pack dairy ice cream, multi-pack water ice cream, take-home ice cream, take-home dairy ice cream, ice cream desserts, bulk ice cream, take-home water ice cream, frozen yoghurt, artisanal ice cream, dairy products, milk, fresh/pasteurised milk, full fat fresh/pasteurised milk, semi skimmed fresh/pasteurised milk, long-life/uht milk, fill fat long life/uht milk, semi skimmed long life/uht milk, fat-free long life/uht milk, goat milk, condensed/evaporated milk, plain condensed/evaporated milk, flavoured, functional and other condensed milk, flavoured milk drinks, dairy only flavoured milk drinks, flavoured milk drinks with fruit juice, soy milk, sour milk drinks, fermented dairy drinks, coffee whiteners, powder milk, flavoured powder milk drinks, cream, cheese, processed cheese, spreadable processed cheese, unspreadable processed cheese, unprocessed cheese, spreadable unprocessed cheese, hard cheese, packaged hard cheese, unpackaged hard cheese, yoghurt, plain/natural yoghurt, flavoured yoghurt, fruited yoghurt, probiotic yoghurt, drinking yoghurt, regular drinking yoghurt, probiotic drinking yoghurt, chilled and shelf-stable desserts, dairy-based desserts, soy-based desserts, chilled snacks, fromage frais and quark, plain fromage frais and quark, flavoured fromage frais and quark, savoury fromage frais and quark, sweet and savoury snacks, fruit snacks, chips/crisps, extruded snacks, tortilla/corn chips, popcorn, pretzels, nuts, other sweet and savoury snacks, snack bars, granola bars, breakfast bars, energy bars, fruit bars, other snack bars, meal replacement products, slimming products, convalescence drinks, ready meals, canned ready meals, frozen ready meals, dried ready meals, chilled ready meals, dinner mixes, frozen pizza, chilled pizza, soup, canned soup, dehydrated soup, instant soup, chilled soup, uht soup, frozen soup, pasta, canned pasta, dried pasta, chilled/fresh pasta, noodles, plain noodles, instant noodles, cups/bowl instant noodles, pouch instant noodles, chilled noodles, snack noodles, canned food, canned meat and meat products, canned fish/seafood, canned vegetables, canned tomatoes, canned beans, canned fruit, canned ready meals, canned soup, canned pasta, other canned foods, frozen food, frozen processed red meat, frozen processed poultry, frozen processed fish/seafood, frozen processed vegetables, frozen meat substitutes, frozen potatoes, oven baked potato chips, other oven baked potato products, non-oven frozen potatoes, frozen bakery products, frozen desserts, frozen ready meals, frozen pizza, frozen soup, frozen noodles, other frozen food, dried food, dessert mixes, dried ready meals, dehydrated soup, instant soup, dried pasta, plain noodles, instant noodles, cups/bowl instant noodles, pouch instant noodles, chilled food, chilled processed meats, chilled fish/seafood products, chilled processed fish, chilled coated fish, chilled smoked fish, chilled lunch kit, chilled ready meals, chilled pizza, chilled soup, chilled/fresh pasta, chilled noodles, oils and fats, olive oil, vegetable and Seed oil, cooking fats, butter, margarine, spreadable oils and fats, functional spreadable oils and fats, sauces, dressings and condiments, tomato pastes and purees, bouillon/stock cubes, stock cubes, gravy granules, liquid stocks and fonds, herbs and spices, seasonings and seasoning blends, fermented sauces, soy based sauces, pasta sauces, wet sauces, dry sauces/powder mixes, ketchup, mayonnaise, regular mayonnaise, mustard, salad dressings, regular salad dressings, low fat salad dressings, vinaigrettes, dips, pickled products, other sauces, dressings and condiments, baby food, milk formula, standard milk formula, follow-on milk formula, toddler milk formula, hypoallergenic milk formula, prepared baby food, dried baby food, other baby food, spreads, jams and preserves, honey, chocolate spreads, nut-based spreads, and yeast-based spreads.

Preferably, the compounds of Formula (I) or (II) can be used to modify or enhance the savory flavor of one or more of the following sub-genuses of comestible compositions: confectioneries, bakery products, ice creams, dairy products, savory snacks, snack bars, meal replacement products, ready meals, soups, pastas, noodles, canned foods, frozen foods, dried foods, chilled foods, oils and fats, baby foods, or spreads, or a mixture thereof.

In general an ingestible composition will be produced that contains a sufficient amount of at least one compound within the scope of Formula (I) or (II) or its various subgenuses described hereinabove to produce a composition having the desired flavor or taste characteristics such as “savory” taste characteristics.

Typically at least a savory flavor modulating amount, of one or more of the compounds of Formula (I) or (II) will be added to the comestible product, so that the savory flavor modified comestible product has an increased savory taste as compared to the comestible product prepared without the compound of Formula (I) or (II), as judged by human beings or animals in general, or in the case of formulations testing, as judged by a majority of a panel of at least eight human taste testers, via procedures described elsewhere herein.

The concentration of savory flavoring agent needed to modulate or improve the flavor of the comestible product or composition will of course vary dependent on many variables, including the specific type of ingestible composition, what savory compounds are already present and the concentrations thereof, the amount of MSG already present, and the enhancer effect of the particular compound on such savory compounds. As noted, a significant application of the compounds of Formula (I) or (II) is for modulating (inducing, enhancing or inhibiting) the savory tastes or other taste properties of other natural or synthetic savory tastants, especially MSG. A broad range of concentrations of the compounds of Formula (I) or (II) can be employed to provide such savory taste enhancement, i.e. from about 0.001 ppm to 100 ppm, or narrower alternative ranges from about 0.1 ppm to about 10 ppm, from about 0.01 ppm to about 30 ppm, from about 0.05 ppm to about 15 ppm, from about 0.1 ppm to about 5 ppm, or from about 0.1 ppm to about 3 ppm.

A variety of classes, subclasses and species of foods are known. Examples of food and beverage products or formulations include, but are not limited to sweet coatings, frostings, or glazes for comestible products or any entity included in the Soup category, the Dried Processed Food category, the Beverage category, the Ready Meal category, the Canned or Preserved Food category, the Frozen Processed Food category, the Chilled Processed Food category, the Snack Food category, the Baked Goods category, the Confectionary category, the Dairy Product category, the Ice Cream category, the Meal Replacement category, the Pasta and Noodle category, and the Sauces, Dressings, Condiments category, the Baby Food category, and/or the Spreads category.

In general, the Soup category refers to canned/preserved, dehydrated, instant, chilled, UHT and frozen soup. For the purpose of this definition soup(s) means a food prepared from meat, poultry, fish, vegetables, grains, fruit and other ingredients, cooked in a liquid which may include visible pieces of some or all of these ingredients. It may be clear (as a broth) or thick (as a chowder), smooth, pureed or chunky, ready-to-serve, semi-condensed or condensed and may be served hot or cold, as a first course or as the main course of a meal or as a between meal snack (sipped like a beverage). Soup may be used as an ingredient for preparing other meal components and may range from broths (consommé) to sauces (cream or cheese-based soups).

“Dehydrated and Culinary Food Category” usually means: (i) Cooking aid products such as: powders, granules, pastes, concentrated liquid products, including concentrated bouillon, bouillon and bouillon like products in pressed cubes, tablets or powder or granulated form, which are sold separately as a finished product or as an ingredient within a product, sauces and recipe mixes (regardless of technology); (ii) Meal solutions products such as: dehydrated and freeze dried soups, including dehydrated soup mixes, dehydrated instant soups, dehydrated ready-to-cook soups, dehydrated or ambient preparations of ready-made dishes, meals and single serve entrees including pasta, potato and rice dishes; and (iii) Meal embellishment products such as: condiments, marinades, salad dressings, salad toppings, dips, breading, batter mixes, shelf stable spreads, barbecue sauces, liquid recipe mixes, concentrates, sauces or sauce mixes, including recipe mixes for salad, sold as a finished product or as an ingredient within a product, whether dehydrated, liquid or frozen.

The Beverage category usually means beverages, beverage mixes and concentrates, including but not limited to, carbonated and non-carbonated beverages, alcoholic and non-alcoholic beverages, ready to drink beverages, liquid concentrate formulations for preparing beverages such as sodas, and dry powdered beverage precursor mixes. The Beverage category also includes the alcoholic drinks, the soft drinks, sports drinks, isotonic beverages, and hot drinks. The alcoholic drinks include, but are not limited to beer, cider/perry, FABs, wine, and spirits. The soft drinks include, but are not limited to carbonates, such as colas and non-cola carbonates; fruit juice, such as juice, nectars, juice drinks and fruit flavored drinks; bottled water, which includes sparkling water, spring water and purified/table water; functional drinks, which can be carbonated or still and include sport, energy or elixir drinks; concentrates, such as liquid and powder concentrates in ready to drink measure. The hot drinks include, but are not limited to coffee, such as fresh, instant, and combined coffee; tea, such as black, green, white, oolong, and flavored tea; and other hot drinks including flavor-, malt- or plant-based powders, granules, blocks or tablets mixed with milk or water.

The Snack Food category generally refers to any food that can be a light informal meal including, but not limited to Sweet and savory snacks and snack bars. Examples of snack food include, but are not limited to fruit snacks, chips/crisps, extruded snacks, tortilla/corn chips, popcorn, pretzels, nuts and other sweet and savory snacks. Examples of snack bars include, but are not limited to granola/muesli bars, breakfast bars, energy bars, fruit bars and other snack bars.

The Baked Goods category generally refers to any edible product the process of preparing which involves exposure to heat or excessive sunlight. Examples of baked goods include, but are not limited to bread, buns, cookies, muffins, cereal, toaster pastries, pastries, waffles, tortillas, biscuits, pies, bagels, tarts, quiches, cake, any baked foods, and any combination thereof.

The Ice Cream category generally refers to frozen dessert containing cream and sugar and flavoring. Examples of ice cream include, but are not limited to: impulse ice cream; take-home ice cream; frozen yoghurt and artisanal ice cream; soy, oat, bean (e.g., red bean and mung bean), and rice-based ice creams.

The Confectionary category generally refers to edible product that is sweet to the taste. Examples of confectionary include, but are not limited to candies, gelatins, chocolate confectionery, sugar confectionery, gum, and the likes and any combination products.

The Meal Replacement category generally refers to any food intended to replace the normal meals, particularly for people having health or fitness concerns. Examples of meal replacement include, but are not limited to slimming products and convalescence products.

The Ready Meal category generally refers to any food that can be served as meal without extensive preparation or processing. The ready meal includes products that have had recipe “skills” added to them by the manufacturer, resulting in a high degree of readiness, completion and convenience. Examples of ready meal include, but are not limited to canned/preserved, frozen, dried, chilled ready meals; dinner mixes; frozen pizza; chilled pizza; and prepared salads.

The Pasta and Noodle category includes any pastas and/or noodles including, but not limited to canned, dried and chilled/fresh pasta; and plain, instant, chilled, frozen and snack noodles.

The Canned/Preserved Food category includes, but is not limited to canned/preserved meat and meat products, fish/seafood, vegetables, tomatoes, beans, fruit, ready meals, soup, pasta, and other canned/preserved foods.

The Frozen Processed Food category includes, but is not limited to frozen processed red meat, processed poultry, processed fish/seafood, processed vegetables, meat substitutes, processed potatoes, bakery products, desserts, ready meals, pizza, soup, noodles, and other frozen food.

The Dried Processed Food category includes, but is not limited to rice, dessert mixes, dried ready meals, dehydrated soup, instant soup, dried pasta, plain noodles, and instant noodles.

The Chill Processed Food category includes, but is not limited to chilled processed meats, processed fish/seafood products, lunch kits, fresh cut fruits, ready meals, pizza, prepared salads, soup, fresh pasta and noodles.

The Sauces, Dressings and Condiments category includes, but is not limited to tomato pastes and purees, bouillon/stock cubes, herbs and spices, monosodium glutamate (MSG), table sauces, soy based sauces, pasta sauces, wet/cooking sauces, dry sauces/powder mixes, ketchup, mayonnaise, mustard, salad dressings, vinaigrettes, dips, pickled products, and other sauces, dressings and condiments.

The Baby Food category includes, but is not limited to milk- or soybean-based formula; and prepared, dried and other baby food.

The Spreads category includes, but is not limited to jams and preserves, honey, chocolate spreads, nut based spreads, and yeast based spreads.

The Dairy Product category generally refers to edible product produced from mammal's milk. Examples of dairy product include, but are not limited to drinking milk products, cheese, yoghurt and sour milk drinks, and other dairy products.

According to the present invention, “non-edible products” include supplements, nutraceuticals, functional food products (e.g., any fresh or processed food claimed to have a health-promoting and/or disease-preventing properties beyond the basic nutritional function of supplying nutrients), pharmaceutical and over the counter products, oral care products such as dentifrices and mouthwashes, cosmetic products such as sweetened lip balms and other personal care products that use sucralose and or other sweeteners.

The term “edible ingredient” herein means any edible component or mixture of components of food or food products, for example the edible ingredients which would typically be found in a recipe for human or animal foods. Edible ingredients include natural and synthetic food components.

In general, over the counter (OTC) product and oral care product generally refer to product for household and/or personal use which may be sold without a prescription and/or without a visit to a medical professional. Examples of the OTC products include, but are not limited to Vitamins and dietary supplements; Topical analgesics and/or anesthetic; Cough, cold and allergy remedies; Antihistamines and/or allergy remedies; and combinations thereof. Vitamins and dietary supplements include, but are not limited to vitamins, dietary supplements, tonics/bottled nutritive drinks, child-specific vitamins, dietary supplements, any other products of or relating to or providing nutrition, and combinations thereof. Topical analgesics and/or anesthetic include any topical creams/ointments/gels used to alleviate superficial or deep-seated aches and pains, e.g. muscle pain; teething gel; patches with analgesic ingredient; and combinations thereof. Cough, cold and allergy remedies include, but are not limited to decongestants, cough remedies, pharyngeal preparations, medicated confectionery, antihistamines and child-specific cough, cold and allergy remedies; and combination products. Antihistamines and/or allergy remedies include, but are not limited to any systemic treatments for hay fever, nasal allergies, insect bites and stings. Examples of oral care product include, but are not limited to mouth cleaning strips, toothpaste, toothbrushes, mouthwashes/dental rinses, denture care, mouth fresheners at-home teeth whiteners and dental floss.

In one embodiment of the present invention, the present compounds, i.e., compounds of Formula (I) or (II) including any subgenus and specific embodiments thereof, can be formulated to make a high-protein composition. By “high-protein composition”, it is meant a composition wherein the percentage of proteins or amino acids is higher than that in natural food or beverage. Examples of the high-protein composition includes, but are not limited to, protein shakes, protein powder, protein-enhanced drinks, and other high protein diet. Examples of the proteins suitable for the high-protein composition include, but are not limited to, vegetable or gluten based proteins, such as wheat, soy, rice, and corn; dairy based proteins such as whey, milk, and caseins; animal based proteins such as poultry, beef, and gelatin; and other protein hydrolysates.

In another embodiment, the present invention provides a flavoring composition comprising a compound of Formula (I) or (II) and a carrier. The term “carrier” refers to a diluent, adjuvant, excipient or vehicle with which the present compound is formulated. Typically, the flavoring composition has a flavor that is stronger or more concentrated than the flavor in a composition directly taken by the mouth. Such a flavoring composition can be added to another composition as a flavor source or to increase the flavor of another composition. That is, the flavoring composition can be used as a seasoning ingredient in another composition. The concentration of the present compound in the flavoring composition is typically higher than that in a ready-to-serve composition. The flavoring composition can be a liquid, semi-liquid, gel, foam, semi-solid, or solid. The solid form may be tablet, powder, granule, cube, capsule, and the like. In one specific embodiment, the flavoring composition also contains MSG or other umami tastants.

Other examples of foods and beverages wherein compounds according to the invention may be incorporated included by way of example carbonated and non-carbonated beverages, e.g., sodas, fruit or vegetable juices, alcoholic and non-alcoholic beverages, confectionary products, e.g., cakes, cookies, pies, candies, chewing gums, gelatins, ice creams, sorbets, puddings, jams, jellies, salad dressings, and other condiments, cereal, and other breakfast foods, canned fruits and fruit sauces and the like.

Additionally, the subject compounds can be used in flavor preparations to be added to foods and beverages. In preferred instances the composition will comprise another flavor or taste modifier such as a savory tastant.

Accordingly, in some embodiments, the inventions relate to methods for modulating the savory taste of a comestible product comprising: a) providing at least one comestible product, or a precursor thereof, and b) combining the comestible product or precursor thereof with at least a savory flavor modulating amount of at least one compound of Formula (I) or (II) or any of its subgenuses, or a comestibly acceptable salt thereof, so as to form a modified comestible product.

The invention also relates to the modified comestible products produced by such processes, and similar processes for producing comestible products well known to those of ordinary skill in the art, especially if such compositions comprise MSG, and the compound is employed as a savory taste enhancer for the MSG also present in the composition.

The compounds of Formula (I) or (II) and its various subgenuses can be combined with or applied to the comestible or medicinal products or precursor thereof in any of innumerable ways known to cooks the world over, or producers of comestible or medicinal products. For example, the compounds of Formula (I) or (II) could be dissolved in or dispersed in or one of many known comestibly acceptable liquids, solids, or other carriers, such as water at neutral, acidic, or basic pH, fruit or vegetable juices, vinegar, marinades, beer, wine, natural water/fat emulsions such as milk or condensed milk, edible oils and shortenings, fatty acids, certain low molecular weight oligomers of propylene glycol, glyceryl esters of fatty acids, and dispersions or emulsions of such hydrophobic substances in aqueous media, salts such as sodium chloride, vegetable flours, solvents such as ethanol, solid edible diluents such as vegetable powders or flours, and the like, and then combined with precursors of the comestible or medicinal products, or applied directly to the comestible or medicinal products.

### Example Procedures for Making the Compounds of Formula (II)

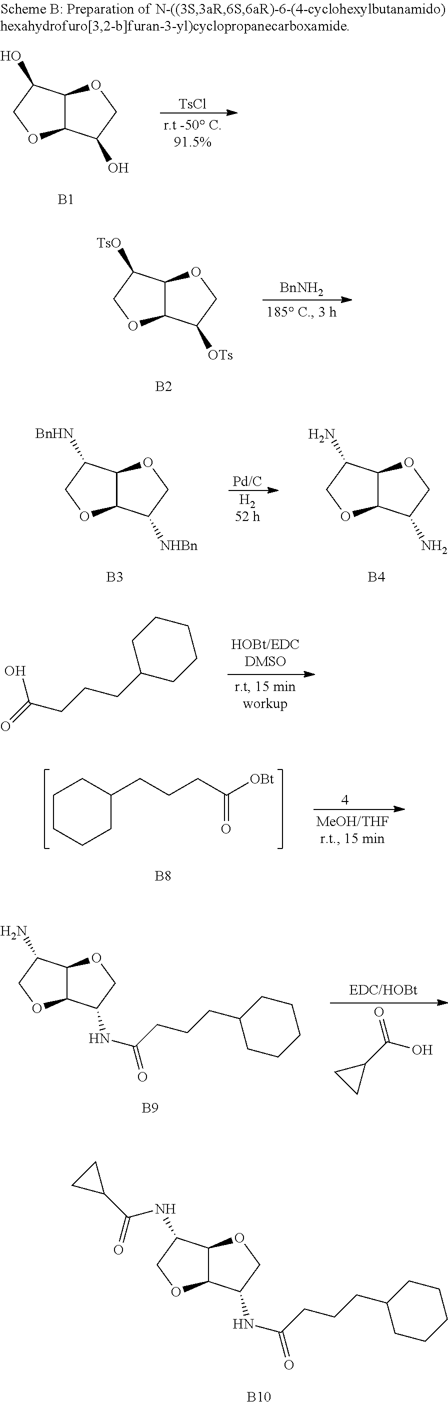

### N-43S,3aR,6S,6aR)-6-(4-Cyclohexylbutanamido)hexahydrofuro[3,2-b]furan-3-yl)cyclopropanecarboxamide (B10)

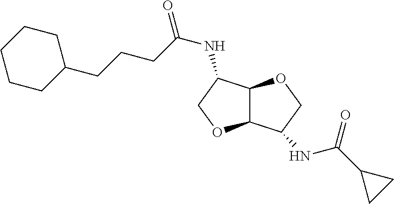

A mixture of N-((3S,3aR,6S,6aR)-6-aminohexahydrofuro[3,2-b]furan-3-yl)-4-cyclohexylbutanamide (B9) (17.5 g, 59.1 mmol), cyclopropylcarboxylic acid (5.1 g, 59.1 mmol), 1-hydroxybenzotriazole (2.0 g, 14.8 mmol) and 1-ethyl-3-[3-dimethylaminopropyl]carbodiimide hydrochloride (14.0 g, 73 mmol) in dry DMSO (60 ml) was stirred for 30 min. Water (350 ml) was added, and the resulting precipitate was filtered. The pure product was obtained by recrystallization from 430 ml of MeCN, and subsequent recrystallization from ethanol to provide N-((3S,3aR,6S,6aR)-6-(4-cyclohexylbutanamido)hexahydrofuro[3,2-b]furan-3-yl)cyclopropanecarboxamide (B10) (14.45 g, 67%). The filtrate was concentrated and chromatographed on silica gel with ethyl acetate as eluent to give an additional 4.42 g of N-((3S,3aR,6S,6aR)-6-(4-cyclohexylbutanamido)hexahydrofuro[3,2-b]furan-3-yl)cyclopropanecarboxamide (B10) (87.7% combined yield). ¹H NMR (400 MHz, CDCl₃) δ 0.73-0.77 (m, 2H), 0.89 (m, 2H), 0.93-0.95 (m, 2H), 1.11-1.25 (m, 6H), 1.36-1.41 (m, 1H), 1.57-1.70 (m, 7H), 2.14 (t, J=8.0 Hz, 2H), 3.80 (m, 2H), 3.93 (m, 2H), 4.44 (m, 2H), 4.53 (m, 2H), 6.09 (d, J=8.0 Hz, 1H), 6.37 (d, J=8.0 Hz, 1H). M+H=365.20.

### N-((3S,3aR,6S,6aR)-6-Aminohexahydrofuro[3,2-b]furan-3-yl)-4-cyclohexylbutanamide (B9)

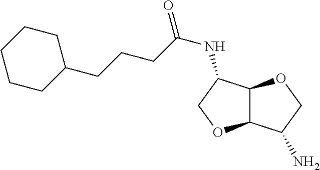

A mixture of cyclohexylbutoic acid (24.34 g, 143 mmol), 1-hydroxybenzotriazole (19.31 g, 143 mmol) and 1-ethyl-3-[3-dimethylaminopropyl]carbodiimide hydrochloride (32.0 g, 167 mmol) in dry DMSO (100 ml) was stirred for 15 min. Water (300 ml) was added, and the solution was extracted with ethyl acetate (400 ml×2), dried over sodium sulfate, filtered and evaporated under reduced pressure below 30° C. until ca 200 ml were left to provide an ethyl acetate solution of intermediate 1H-benzo[d][1,2,3]triazol-1-yl 4-cyclohexylbutanoate (B8).

To a solution of (3S,3aR,6S,6aR)-hexahydrofuro[3,2-b]furan-3,6-diamine (B4) (20.5 g, 142.2 mmol) in 40 ml of methanol and 70 ml of acetonitrile under vigorous stirring was slowly added the above 1H-benzo[d][1,2,3]triazol-1-yl-4-cyclohexylbutanoate (B8) ethyl acetate solution. The reaction was stirred for 15 min, at which time HCl (280 ml, 1N) and ethyl acetate (150 ml) were added, and the mixture was stirred for ca 10 min. The aqueous layer was separated and extracted with ethyl acetate (200 ml). The combined organic phases were washed with 1N HCl (150 ml×4). The combined HCl layers were basified with KOH and K₂CO₃, extracted with ethyl acetate (400 ml×3), dried over sodium sulfate. Filtration and evaporation of solvent gave N-((3S,3aR,6S,6aR)-6-aminohexahydrofuro[3,2-b]furan-3-yl)-4-cyclohexylbutanamide (B9) (17.6 g, 45.5%). ¹H NMR (400 MHz, CD₃OD) δ 0.89 (m, 2H), 1.14-1.25 (m, 6H), 1.56-1.71 (m, 7H), 2.16 (t, J=8.0 Hz, 2H), 3.41 (m, 1H), 3.62 (m, 2H), 3.89 (m, 2H), 4.23 (m, 1H), 4.39 (d, J=4.0 Hz, 1H), 4.54 (d, J=4.0 Hz, 1H). M+H=297.15.

### (3S,3aR,6S,6aR)-Hexahydrofuro[3,2-b]furan-3,6-diamine (B4)

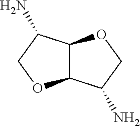

(3S,3aR,6S,6aR)—N3,N6-Dibenzylhexahydrofuro[3,2-b]furan-3,6-diamine (B3) (59 g) was dissolved in 500 ml of methanol under nitrogen. Pd/C (19.5 g, Aldrich, 10%, wet, 50% dry material) was added. The reaction was stirred under two H₂-containing balloons for 24 h, after which time, more catalyst (10.0 g, Aldrich, 10%, wet, 50% dry material) was added and the reaction was continued for an additional 28 h. During this period, hydrogen balloons were refilled 4 times. Upon completion, the reaction mixture was filtered through celite and the solvent was evaporated to give (3S,3aR,6S,6aR)-hexahydrofuro[3,2-b]furan-3,6-diamine (B4) (20.5 g) of crude product. This product was directly used for the next step. ¹H NMR (400 MHz, CD₃OD) δ 3.37 (m, 2H), 3.60 (dd, J=4.0, 4.0 Hz, 2H), 3.81 (d, J=4.0 Hz, 4H), 3.85 (dd, J=8.0, 4.0 Hz, 2H), 4.44 (s, 2H). M+H=145.10.

### (3S,3aR,6S,6aR)—N3,N6-Dibenzylhexahydrofuro[3,2-b]furan-3,6-diamine (B3)

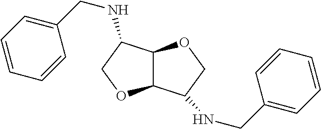

A mixture of (3R,3aS,6R,6aS)-hexahydrofuro[3,2-b]furan-3,6-diyl bis(4-methylbenzenesulfonate) (B2) (59.1 g, 130 mmol) and benzylamine (84 ml, 770.0 mmol) was heated at 185° C. under nitrogen for 3 h. After cooling, it was dissolved in 500 ml of ethyl acetate and was washed with saturated sodium carbonate solution. The aqueous phase was extracted with EtOAc (200 ml). The combined organic layer was washed with brine, dried over sodium sulfate, filtered and evaporated. The residue was dried under high vacuum to remove the unreacted benzylamine at 75-80° C. to provide 3S,3aR,6S,6aR)—N3,N6-dibenzylhexahydrofuro[3,2-b]furan-3,6-diamine (B3) (59 g) as an orange oil. This was used directly for the next step. The analytically pure product was obtained by column chromatography. ¹H NMR (400 MHz, CDCl₃) δ 3.33 (m, 2H), 3.65 (dd, J=8.0, 4.0 Hz, 2H), 3.81 (d, J=4.0 Hz, 4H), 3.90 (dd, J=12.0, 4.0 Hz, 2H), 4.53 (s, 2H), 7.24-7.32 (m, 10H). M+H=325.10.

### (3R,3aS,6R,6aS)-Hexahydrofuro[3,2-b]furan-3,6-diyl bis(4-methylbenzenesulfonate) (B2)

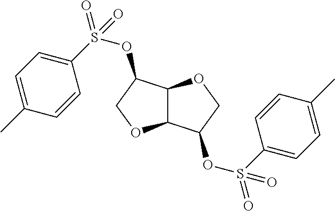

To a solution of isomannide (24.6 g, 130 mmol, Aldrich, 95%) and tosyl chloride (73.0 g, 384 mmol) in 500 ml of dichloromethane at room temperature was slowly added triethylamine (49 ml, 352.2 mmol), and the solution was stirred at r.t. for 20 h. More triethylamine (7 ml) was added and the solution was heated at 50° C. for 5 h. After cooling down, saturated sodium carbonate solution (200 ml) was added, and the reaction was stirred for 10 min. This mixture was transferred to a separatory funnel and ca. 200 ml of dichloromethane was added. The organic phase was washed with brine (1×), dried over sodium sulfate, filtered and evaporated under reduced pressure to give a sticky oil. It was dissolved in 300 ml of methanol and 7 ml of water and heated to boil. The solution was gradually cooled down and triturated to give a precipitate which was collected by a filtration process and washed with a small amount of methanol. This process of precipitation and collection was repeated to the filtrate a couple of times. The combined precipitates give the desired product (3R,3aS,6R,6aS)-hexahydrofuro[3,2-b]furan-3,6-diyl bis(4-methylbenzenesulfonate) (B2) (total 66.5 g, 91.5%) as a white solid. ¹H NMR (400 MHz, CDCl₃) δ 2.44 (s, 6H), 3.72 (m, 2H), 3.90 (m, 2H), 4.47 (d, J=4.0 Hz, 2H), 4.80 (m, 2H), 7.37 (d, J=8.0 Hz, 4H), 7.80 (d, J=8.0 Hz, 4H). M+H=455.15.

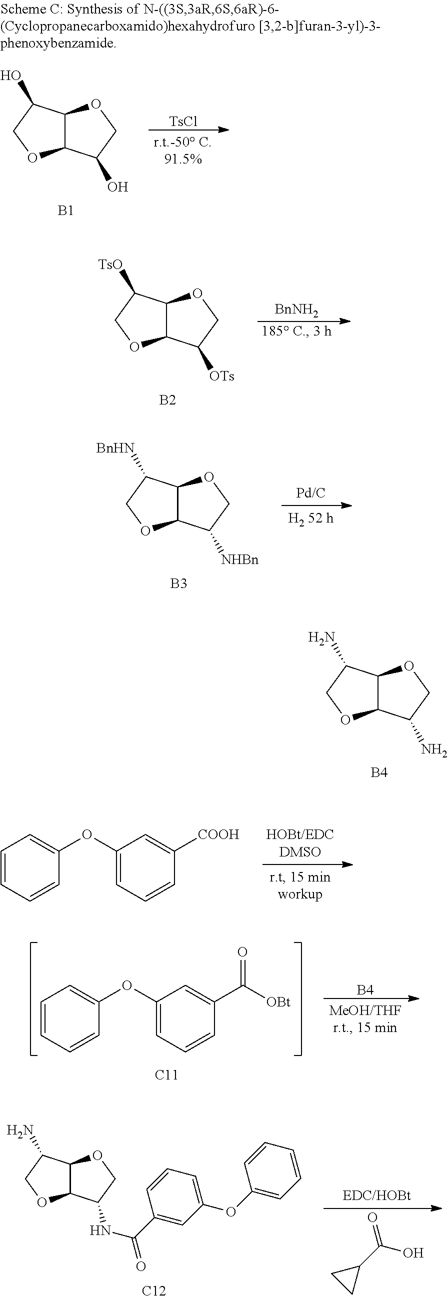

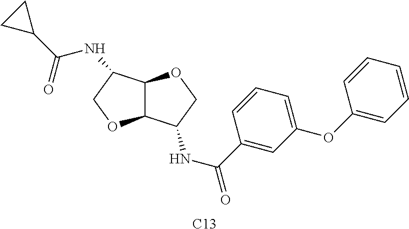

### N-((3S,3aR,6S,6aR)-6-(Cyclopropanecarboxamido)hexahydrofuro[3,2-b]furan-3-yl)-3-phenoxybenzamide (C13)

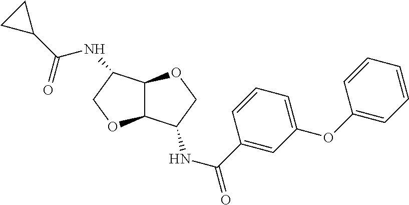

A mixture of N-((3S,3aR,6S,6aR)-6-aminohexahydrofuro[3,2-b]furan-3-yl)-3-phenoxybenzamide C12 (7.3 g, 21.5 mmol), cyclopropylcarboxylic acid (1.85 g, 21.5 mmol), 1-hydroxybenzotriazole (0.6 g, 4.4 mmol) and 1-ethyl-3-[3-dimethylaminopropyl]carbodiimide hydrochloride (5.0 g, 26.2 mmol) in dry DMSO (50 ml) was stirred for 20 min. Water (400 ml) was added, and the resulting precipitate was collected through a filtration process to give 8.6 g of crude product. The residue was purified by column chromatography on silica gel with ethyl acetate as eluent followed by recrystallization from ethanol/water to provide N-((3S,3aR,6S,6aR)-6-(cyclopropanecarboxamido)hexahydrofuro[3,2-b]furan-3-yl)-3-phenoxybenzamide (C13) (7.4 g, 85%) ¹H NMR (400 MHz, DMSO) δ 0.64 (m, 4H), 1.54 (m, 1H), 3.31 (m, 1H), 3.63 (m, 1H), 3.84 (m, 1H), 3.91 (m, 1H), 4.10 (m, 1H), 4.29 (m, 1H), 4.44 (d, J=4.0 Hz, 1H), 4.56 (d, J=4.0 Hz, 1H), 7.01 (d, 2H, J=8.0 Hz,), 7.38 (m, 2H), 7.02 (m, 2H), 7.41 (m, 2H), 7.48 (d, J=8.0 Hz, 1H), 8.35 (d, J=8.0 Hz, 1H), 8.63 (d, J=8.0 Hz, 1H). ¹³C NMR (100 MHz, DMSO) δ 7.10, 14.0, 56.5, 57.4, 72.3, 86.8, 87.0, 118.3, 119.3, 122.3, 123.2, 124.4, 130.7, 136.6, 157.1, 157.3, 166.3, 173.2, M+H=409.

### N-((3S,3aR,6S,6aR)-6-Aminohexahydrofuro[3,2-b]furan-3-yl)-3-phenoxybenzamide (C12)

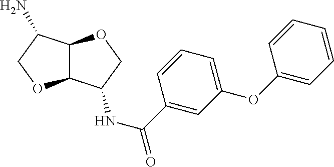

A mixture of 3-phenoxybenzoic acid (12.0 g, 56.0 mmol), 1-hydroxybenzotriazole (7.6 g, 56.3 mmol) and 1-ethyl-3-[3-dimethylaminopropyl]carbodiimide hydrochloride (13.0 g, 68.1 mmol) in dry DMSO (50 ml) was stirred for 15 min. Water (300 ml) was added, and the solution was extracted with ethyl acetate (350 ml×2), washed with brine, dried over sodium sulfate, filtered and evaporated under reduced pressure below 30° C. until ca 80 ml were left.

(3S,3aR,6S,6aR)-hexahydrofuro[3,2-b]furan-3,6-diamine (7.8 g, 54.1 mmol) (B4) was dissolved in 40 ml of methanol and 50 ml of acetonitrile. To this solution was slowly added under vigorous stirring the above 1H-benzo[d][1,2,3]triazol-1-yl 3-phenoxybenzoate (C11) ethyl acetate solution over 10 min. The reaction was continued for an additional 15 min. HCl (120 ml, 1N) was added, and the mixture was extracted with ethyl acetate (300 ml×2). The combined organic phase was washed with 1 N HCl (100 ml×3). The combined HCl washes including the original HCl solution was basified with KOH and K₂CO₃. The solution was extracted with ethyl acetate (400 ml×2), dried over sodium sulfate and filtered. Evaporation of the solvent provided N-((3S,3aR,6S,6aR)-6-aminohexahydrofuro[3,2-b]furan-3-yl)-3-phenoxybenzamide (C12) (7.3 g, 39.6%). ¹H NMR (400 MHz, CD₃OD) δ 3.40 (m, 1H), 3.64 (m, 1H), 3.78 (m, 1H), 3.90 (m, 1H), 3.97 (m, 1H), 4.43 (m, 2H), 4.71 (m, 1H), 6.97 (d, J=8.0 Hz, 2H), 7.10 (m, 2H), 7.32 (m, 2H), 7.48 (m, 1H), 7.38 (t, J=8.0 Hz, 1H), 7.56 (d, 1H, J=8.0 Hz). M+H=341.

### Measuring the Biological Activity of the Compounds of the Invention

In Vitro hT1R1/hT1R3 activation assay:

An HEK293 cell line derivative (See e.g., Chandrashekar, et al., Cell (2000) 100: 703-711) which stably expresses Gα15 and hT1R1/hT1R3 under an inducible promoter (see WO 03/001876 A2) was used to identify compounds with umami tasting properties.

Compounds of the present invention were initially selected based on their activity on the hT1R1/hT1R3-HEK293-Gα15 cell line. Activity was determined using an automated fluorometric imaging assay on a FLIPR instrument (Fluorometric Intensity Plate Reader, Molecular Devices, Sunnyvale, Calif.) (designated FLIPR assay). Cells from one clone (designated clone I-17) were seeded into 384-well plates (at approximately 48,000 cells per well) in a medium containing Dulbecco's modified Eagle's medium (DMEM) supplemented with GlutaMAX (Invitrogen, Carlsbad, Calif.), 10% dialyzed fetal bovine serum (Invitrogen, Carlsbad, Calif.), 100 Units/ml Penicillin G, 100 μg/ml Streptomycin (Invitrogen, Carlsbad, Calif.) and 60 pM mifepristone (to induce expression of hT1R1/hT1R3, (see WO 03/001876 A2). I-17 cells were grown for 48 hours at 37° C. I-17 cells were then loaded with the calcium dye Fluo-3AM (Molecular Probes, Eugene, Oreg.), 4 μM in a phosphate buffered saline (D-PBS) (Invitrogen, Carlsbad, Calif.), for 1.5 hours at room temperature. After replacement with 25 μl D-PBS, stimulation was performed in the FLIPR instrument and at room temperature by the addition of 25 μl D-PBS supplemented with different stimuli at concentrations corresponding to twice the desired final level. Receptor activity was quantified by determining the maximal fluorescence increases (using a 480 nm excitation and 535 nm emission) after normalization to basal fluorescence intensity measured before stimulation.

For dose-responses analysis, stimuli were presented in duplicates at 10 different concentrations ranging from 1.5 nM to 30 μM. Activities were normalized to the response obtained with 60 mM monosodium glutamate, a concentration that elicits maximum receptor response. EC₅₀s (concentration of compound that causes 50% activation of receptor) were determined using a non-linear regression algorithm, where the Hill slope, bottom asymptotes and top asymptotes were allow to vary. Identical results were obtained when analyzing the dose-response data using commercially available software for non-linear regression analysis such as GraphPad PRISM (San Diego, Calif.). All compounds listed in all of the Examples herein have been tested and each have an EC₅₀ of 30 μM or less.

In order to determine the dependency of hT1R1/hT1R3 for the cell response to different stimuli, selected compounds were subjected to a similar analysis on I-17 cells that had not been induced for receptor expression with mifepristone (designated as un-induced I-17 cells). The un-induced I-17 cells do not show any functional response in the FLIPR assay to monosodium glutamate or other umami-tasting substances. Compounds were presented to un-induced umami cells at 10 μM—or three times the maximum stimulation used in the dose-response analysis. Compounds covered in this document do not show any functional response when using un-induced umami cells in the FLIPR assay.

Upon testing, the compounds of the present invention showed an EC₅₀ for the hT1R1/hTIR3 receptor of less than about 10 μM, 5 μM, 3 μM, 2 μM, 1 μM, or 0.5 μM.

### Time-Intensity Testing Protocol

Overview: Panelists evaluate various solutions for umami intensity over time.
General procedure: Samples presented to panelists include various concentrations of MSG and compounds of interest. Samples may contain 0.1% ethanol to assist in dissolving compounds into solution. Samples are three-digit blind coded and served in 20 ml volumes. Samples are presented to panelists in randomized, counterbalanced order. All data is collected using the Compusense five software program. Umami intensity is rated on a scale from 0 to 100 (0=no umami intensity; 100=strong umami intensity). Panelists sip a sample and begin rating umami intensity. Panelists move the mouse on the horizontal scale to correspond to the umami intensity they perceive. Panelists continue rating the intensity for 70 seconds, and then expectorate the sample. The software program records an intensity score for each panelist for each 1 second period during the sample evaluation.

After tasting each sample, panelists sip and swish water in the mouth for a few seconds and expectorate. They may repeat this several times. Panelists wait 30 minutes before tasting the next sample.

Data analysis: Time-intensity curves are created for each of the samples and various parameters of the test are compared statistically.

To create the graphical representation of the sample: For each of the 70 seconds of the evaluation, panelists individual scores are averaged and presented graphically. For example, each panelist's intensity score at time=0 seconds is averaged and plotted. The same calculation is made for each of the 70 seconds to create the time-intensity curve.

To evaluate the Time to Maximum Intensity parameter: A time-intensity curve is created for each panelist. Each panelist's graph shows their individual point of maximum umami intensity, and the specific time to maximum is recorded. The specific time scores for each panelist are averaged. Next, the average scores for the various samples are compared using ANOVA, and multiple comparisons are performed using Tukey's Honestly Significant Difference test at alpha=0.05.

### EXAMPLES

The following examples are given to illustrate a variety of exemplary embodiments of the invention and are not intended to be limiting in any manner.

### Example 1

### N-((3S,3aR,6S,6aR)-6-(cyclopropanecaboxamido)hexahydrofuro[3,2-b]furan-3-yl)-3-(4-ethoxyphenoxy)benzamide

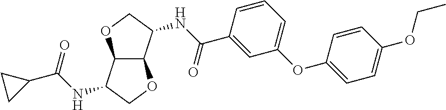

To a solution of 3-(4-ethoxyphenoxy)benzoic acid (6.5 mg, 25 umol) (Example 10, HOBt (3.4 mg, 25 umol), and EDCI (4.8 mg, 25 umol) in 500 uL DMF was added N-((3S,3aR,6S,6aR)-6-aminohexahydrofuro[3,2-b]furan-3-yl)cyclopropanecarboxamide (5.3 mg, 25 umol) (Example 1a). The solution was stirred at room temperature overnight. The crude was purified on mass triggered HPLC (10% to 90% acetonitrile in water), prep-TLC (MeOH/DCM), then re-crystallized from EtOH/water to yield N-((3S,3aR,6S,6aR)-6-(cyclopropanecarboxamido)hexahydrofuro[3,2-b]furan-3-yl)-3-(4-ethoxyphenoxy)benzamide (6.0 mg, 53.1%). M+H=453.

### Example 1a

### N-((3S,3aR,6S,6aR)-6-aminohexahydrofuro[3,2-b]furan-3-yl)cyclopropanecarboxamide

To a solution of N-((3S,3aR,6S,6aR)-6-azidohexahydrofuro[3,2-b]furan-3-yl)cyclopropanecarboxamide (1.19 g, 5 mmol) (Example 1b) in 25 mL MeOH, was added 10% palladium on carbon (53 mg). The solution was stirred under H₂ overnight at room temperature. The solution was then filtered, and the MeOH was evaporated to yield N-((3S,3aR,6S,6aR)-6-aminohexahydrofuro[3,2-b]furan-3-yl)cyclopropanecarboxamide (1.06 g, quantitative) as pure product. M+H=213.

### Example 1b

### N-((3S,3aR,6S,6aR)-6-azidohexahydrofuro[3,2-b]furan-3-yl)cyclopropanecarboxamide

To a solution of cyclopropane carboxylic acid (1.26 mL, 20 mmol), HOBt (2.7 g, 20 mmol), and EDCI (3.8 g, 20 mmol) in 60 mL DMF was added (3S,3aR,6S,6aR)-6-azidohexahydrofuro[3,2-b]furan-3-amine (3.4 g, 20 mmol) (Example 1c), and the mixture was stirred at room temperature overnight. Upon completion, the reaction was concentrated and purified on mass triggered HPLC (10% to 90% acetonitrile in water) to yield N-((3S,3aR,6S,6aR)-6-azidohexahydrofuro[3,2-b]furan-3-yl)cyclopropanecarboxamide (3.29 g, 69%). M+H=239.

### Example 1c

### (3S,3aR,6S,6aR)-6-azidohexahydrofuro[3,2-b]furan-3-amine

To a solution of (3S,3aR,6S,6aR)-3,6-diazidohexahydrofuro[3,2-b]furan (7.84 g, 40 mmol) (Example 1d) in 50 mL EtOAc/Et₂O (1:1) and 40 mL 5% HCl solution in water, was added Ph₃P (10.5 g, 40 mmol) in small portions at 0° C. over 1 hour. The mixture was then stirred at room temperature overnight. The organic layer was discarded and the aqueous was washed 2× with methylene chloride. The aqueous layer was then carefully basified to pH 9 using a 1N NaOH solution. The solution was then extracted with methylene chloride 3×, dried over Na₂CO₃ and filtered. The methylene chloride was evaporated to of (3S,3aR,6S,6aR)-6-azidohexahydrofuro[3,2-b]furan-3-amine yield (4.1 g, 60.2%) as a yellowish liquid. M+H=171.

### Example 1d

### (3S,3aR,6S,6aR)-3,6-diazidohexahydrofuro[3,2-b]furan

To a solution of (3R,3aS,6R,6aS)-hexahydrofuro[3,2-h]furan-3,6-diyl bis(trifluoromethanesulfonate) (Example 1e) (20 g, 48.75 mmol) in DMF (250 mL), was added NaN₃ (9.8 g, 151 mmol). The reaction mixture was heated to 120° C. for 2 h, cooled to rt and concentrated in vacuo. The residue was diluted with CH₂Cl₂, and the solid salts were removed by filtration. The filtrate was washed with H₂O and brine, dried over MgSO₄, filtered and concentrated to provide (3S,3aR,6S,6aR)-3,6-diazidohexahydrofuro[3,2-b]furan (9.8 g, quant.) as a light brown oil. ¹H NMR (CDCl₃ 400 MHz): 3.90 (dd, J=10.4, 4.2 Hz, 2H), 3.95 (dd, J=10.3, 1.5 Hz, 2H), 4.08 (m, 2H), 4.63 (s, 2H).

### Example 1e

### (3R,3aS,6R,6aS)-hexahydrofuro[3,2-b]furan-3,6-diyl bis(trifluoromethanesulfonate)

To a solution of (3R,3aR,6R,6aR)-hexahydrofuro[3,2-b]furan-3,6-diol (52.6 g, 342 mmol) and pyridine (66 mL, 821 mmol) in CH₂Cl₂ (800 mL) at 0° C., was added triflic anhydride (135 mL, 821 mmol) dropwise. After complete addition, the reaction was slowly warmed to rt, and stirred under N₂ overnight. Upon completion, the reaction mixture was diluted with CH₂Cl₂, washed with 1N HCl, water, sat'd NaHCO₃, water and brine. The organic layer was dried over MgSO₄, filtered and concentrated to provide (3R,3aS,6R,6aS)-hexahydrofuro[3,2-b]furan-3,6-diyl bis(trifluoromethanesulfonate) (140.5 g, quant.) as an off-white solid. ¹H NMR (CDCl₃ 400 MHz): 4.17 (d, J=5.0 Hz, 4H), 4.78 (m, 2H), 5.23 (m, 2H), 4.63 (s, 2H).

### Example 1f

### 3-(4-ethoxyphenoxy)benzoic acid

To ethyl 3-(4-ethoxyphenoxy)benzoate (286 mg, 1 mmol) (Example 1g) was added 10 mL of THF/MeOH (1:1) and 10 mL of NaOH 1N solution. This solution was stirred at room temperature for 4 hours. The mixture was then acidified using 1N HCl solution. The product was then extracted with EtOAc (3×), dried over sodium sulfate, and concentrated to yield 3-(4-ethoxyphenoxy)benzoic acid (196 mg, 76.0%) as a pure product.

### Example 1g

### Ethyl 3-(4-ethoxyphenoxy)benzoate

To a solution of ethyl 3-hydroxybenzoate (322 mg, 2.0 mmol) and potassium carbonate (461 mg, 3.52 mmol) in 5 mL acetone was added 1-bromo-4-ethoxybenzene (505 uL, 3.52 mmol). The solution was refluxed overnight. The solution was then filtered and concentrated to yield ethyl 3-(4-ethoxyphenoxy)benzoate (296 mg, 51.7%) pure enough to carry on to the next step.

### Example 2

### N-((3S,3aR,6S,6aR)-6-benzamidohexahydrofuro[3,2-b]furan-3-yl)-3-phenoxybenzamide

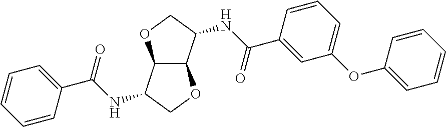

Prepared as in Example 1 from benzoic acid and N-((3S,3aR,6S,6aR)-6-aminohexahydrofuro[3,2-b]furan-3-yl)-3-phenoxybenzamide (Example 2a) to yield N-((3S,3aR,6S,6aR)-6-benzamidohexahydrofuro[3,2-b]furan-3-yl)-3-phenoxybenzamide (4.9 mg, 44.1%). M+H=445.

### Example 2a

### N-((3S,3aR,6S,6aR)-6-aminohexahydrofuro[3,2-b]furan-3-yl)-3-phenoxybenzamide

Prepared as in Example 1a from N-((3S,3aR,6S,6aR)-6-azidohexahydrofuro[3,2-b]furan-3-yl)-3-phenoxybenzamide (Example 2b) to yield N-((3S,3aR,6S,6aR)-6-aminohexahydrofuro[3,2-b]furan-3-yl)-3-phenoxybenzamide (1.75 g, 47.8%). M+H=341.

### Example 2b

### N-((3S,3aR,6S,6aR)-6-azidohexahydrofuro[3,2-b]furan-3-yl)-3-phenoxybenzamide

Prepared as in example 1b from 3-phenoxybenzoic acid and (3S,3aR,6S,6aR)-6-azidohexahydrofuro[3,2-b]furan-3-amine (Example 1c) to yield N-((3S,3aR,6S,6aR)-6-azidohexahydrofuro[3,2-b]furan-3-yl)-3-phenoxybenzamide (1.75 g, 47.8%). M+H=367.

### Example 3

### N-((3S,3aR,6S,6aR)-6-(cyclopropanecaboxamido)hexahydrofuro[3,2-b]furan-3-yl)-3-propoxybenzamide

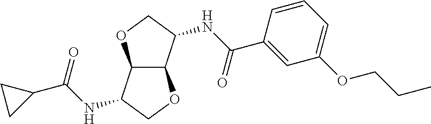

Prepared as in Example 1 from 3-propoxybenzoic acid (Example 3a) and N-((3S,3aR,6S,6aR)-6-aminohexahydrofuro[3,2-b]furan-3-yl)cyclopropanecarboxamide (Example 1a) to yield N-((3S,3aR,6S,6aR)-6-(cyclopropanecaboxamido) hexahydrofuro[3,2-b]furan-3-yl)-3-propoxybenzamide (4.3 mg, 45.9%). M+H=375.

### Example 3a

### 3-propoxybenzoic acid

Prepared as in example if from ethyl 3-propoxybenzoate (Example 3b) to yield 3-propoxybenzoic acid.

### Example 3b

### Ethyl 3-propoxybenzoate

Prepared as in example 1g from ethyl 3-hydroxybenzoate and 1-bromopropane to yield ethyl 3-propoxybenzoate.

### Example 4

### N-((3S,3aR,6S,6aR)-6-(cyclopropanecaboxamido)hexahydrofuro[3,2-b]furan-3-yl)-3-isopropoxybenzamide

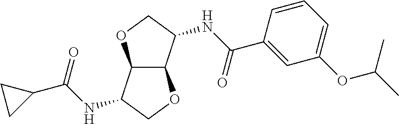

Prepared as in Example 1 from 3-isopropoxybenzoic acid (Example 4a) and N-((3S,3aR,6S,6aR)-6-aminohexahydrofuro[3,2-b]furan-3-yl)cyclopropanecarboxamide (Example 1a) to yield N-((3S,3aR,6S,6aR)-6-(cyclopropanecaboxamido) hexahydrofuro[3,2-b]furan-3-yl)-3-isopropoxybenzamide (4.3 mg, 45.9%). M+H=375.

### Example 4a

### 3-isopropoxybenzoic acid

Prepared as in example if from ethyl 3-isopropoxybenzoate (Example 4b) to yield 3-isopropoxybenzoic acid.

### Example 4b

### Ethyl 3-isopropoxybenzoate

Prepared as in example 1g from ethyl 3-hydroxybenzoate and 2-bromopropane to yield ethyl 3-isopropoxybenzoate.

### Example 5

### 3-(cyclopentyloxy)-N-((3S,3aR,6S,6aR)-6-(cyclopropanecaboxamido)hexahydrofuro[3,2-b]furan-3-yl)benzamide

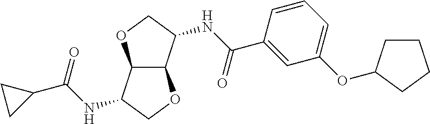

Prepared as in example 1 from 3-(cyclopentyloxy)benzoic acid (Example 5a) and N-((3S,3aR,6S,6aR)-6-aminohexahydrofuro[3,2-b]furan-3-yl)cyclopropanecarboxamide (Example 1a) to yield 3-(cyclopentyloxy)-N-((3S,3aR,6S,6aR)-6-(cyclopropanecaboxamido)hexahydrofuro[3,2-b]furan-3-yl)benzamide (4.9 mg, 48.9%). M+H=401.

### Example 5a

### 3-(cyclopentyloxy)benzoic acid

Prepared as in example if from ethyl 3-(cyclopentyloxy)benzoate (Example 5b) to yield 3-(cyclopentyloxy)benzoic acid.

### Example 5b

### Ethyl 3-(cyclopentyloxy)benzoate

Prepared as in example 1 g from ethyl 3-hydroxybenzoate and bromocyclopentane to yield ethyl 3-(cyclopentyloxy)benzoate.

### Example 6

### N-((3S,3aR,6S,6aR)-6-(3-phenoxybenzamido)hexahydrofuro[3,2-b]furan-3-yl)picolinamide

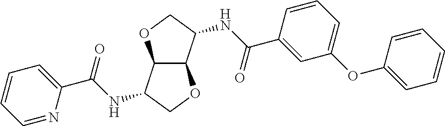

Prepared as in example 1 from picolinic acid and N-((3S,3aR,6S,6aR)-6-aminohexahydrofuro[3,2-b]furan-3-yl)-3-phenoxybenzamide (Example 2a) to yield N-((3S,3aR,6S,6aR)-6-(3-phenoxybenzamido)hexahydrofuro[3,2-b]furan-3-yl)picolinamide (4.1 mg, 36.8%). M+H=446.

### Example 7

### N-((3S,3aR,6S,6aR)-6-(3-phenoxybenzamido)hexahydrofuro[3,2-b]furan-3-yl)nicotinamide

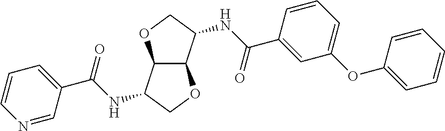

Prepared as in example 1 from nicotinic acid and N-((3S,3aR,6S,6aR)-6-aminohexahydrofuro[3,2-b]furan-3-yl)-3-phenoxybenzamide (Example 2a) to yield N-((3S,3aR,6S,6aR)-6-(3-phenoxybenzamido)hexahydrofuro[3,2-b]furan-3-yl)nicotinamide (4.1 mg, 36.8%). M+H=446.

### Example 8

### 3-hydroxy-N-((3S,3aR,6S,6aR)-6-(3-phenoxybenzamido)hexahydrofuro[3,2-b]furan-3-yl)benzamide

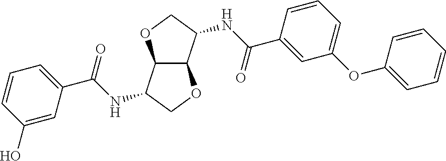

Prepared as in example 1 from 3-hydroxybenzoic acid and N-((3S,3aR,6S,6aR)-6-aminohexahydrofuro[3,2-b]furan-3-yl)-3-phenoxybenzamide (Example 2a) to yield 3-hydroxy-N-((3S,3aR,6S,6aR)-6-(3-phenoxybenzamido)hexahydrofuro[3,2-b]furan-3-yl)benzamide (4.7 mg, 40.8%). M+H=461.

### Example 9

### N-((3S,3aR,6S,6aR)-6-(4-hydroxybenzamido)hexahydrofuro[3,2-b]furan-3-yl)-3-phenoxybenzamide

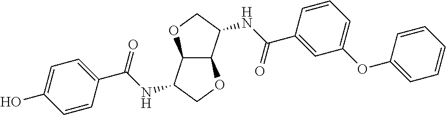

Prepared as in example 1 from 4-hydroxybenzoic acid and N-((3S,3aR,6S,6aR)-6-aminohexahydrofuro[3,2-b]furan-3-yl)-3-phenoxybenzamide (Example 2a) to yield N-((3S,3aR,6S,6aR)-6-(4-hydroxybenzamido)hexahydrofuro[3,2-h]furan-3-yl)-3-phenoxybenzamide (4.7 mg, 40.8%). M+H=461.

### Example 10

### 3-methyl-N-((3S,3aR,6S,6aR)-6-(3-phenoxybenzamido) hexahydrofuro[3,2-b]furan-3-yl)-benzamide

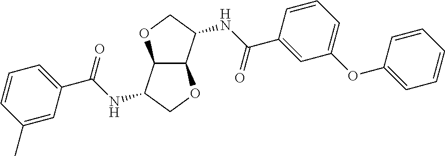

Prepared as in example 1 from 3-methylbenzoic acid and N-((3S,3aR,6S,6aR)-6-aminohexahydrofuro[3,2-b]furan-3-yl)-3-phenoxybenzamide (Example 2a) to yield 3-methyl-N-((3S,3aR,6S,6aR)-6-(3-phenoxybenzamido)hexahydrofuro[3,2-b]furan-3-yl)-benzamide (4.4 mg, 38.4%). M+H=459.

### Example 11

### N-((3S,3aR,6S,6aR)-6-acetamidohexahydrofuro[3,2-b]furan-3-yl)-3-phenoxybenzamide

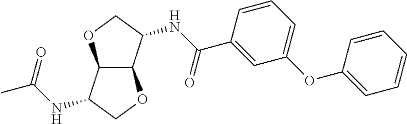

Prepared as in example 1 from acetic acid and N-((3S,3aR,6S,6aR)-6-aminohexahydrofuro[3,2-b]furan-3-yl)-3-phenoxybenzamide (Example 2a) to yield N-((3S,3aR,6S,6aR)-6-acetamidohexahydrofuro[3,2-b]furan-3-yl)-3-phenoxybenzamide (3.7 mg, 38.7%). M+H=383.

### Example 12

### 3-phenoxy-N-((3S,3aR,6S,6aR)-6-propionamidohexahydrofuro[3,2-b]furan-3-yl)-benzamide

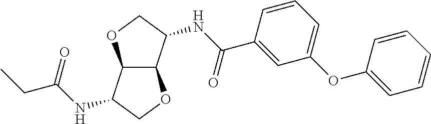

Prepared as in example 1 from propionic acid and N-((3S,3aR,6S,6aR)-6-aminohexahydrofuro[3,2-b]furan-3-yl)-3-phenoxybenzamide (Example 2a) to yield 3-phenoxy-N-((3S,3aR,6S,6aR)-6-propionamidohexahydrofuro[3,2-b]furan-3-yl)-benzamide (3.6 mg, 36.4%). M+H=397.

### Example 13

### 3-methyl-N-((3S,3aR,6S,6aR)-6-(3-phenoxybenzamido)hexahydrofuro[3,2-b]furan-3-yl)-benzamide

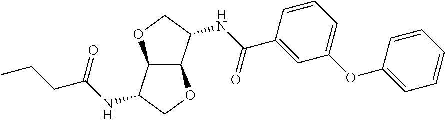

Prepared as in example 1 from butyric acid and N-((3S,3aR,6S,6aR)-6-aminohexahydrofuro[3,2-b]furan-3-yl)-3-phenoxybenzamide (Example 2a) to yield N-((3S,3aR,6S,6aR)-6-butyramidohexahydrofuro[3,2-b]furan-3-yl)-3-phenoxybenzamide (4.1 mg, 40.0%). M+H=411.

### Example 14

### N-((3S,3aR,6S,6aR)-6-(cyclobutanecarboxamido)hexahydrofuro[3,2-b]furan-3-yl)-3-phenoxybenzamide

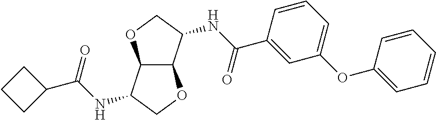

Prepared as in example 1 from cyclobutanecarboxylic acid and N-((3S,3aR,6S,6aR)-6-aminohexahydrofuro[3,2-b]furan-3-yl)-3-phenoxybenzamide (Example 2a) to yield N-((3S,3aR,6S,6aR)-6-(cyclobutanecarboxamido)hexahydrofuro[3,2-b]furan-3-yl)-3-phenoxybenzamide (4.1 mg, 38.9%). M+H=423.

### Example 15

### N-((3S,3aR,6S,6aR)-6-(cyclopentanecarboxamido)hexahydrofuro[3,2-b]furan-3-yl)-3-phenoxybenzamide

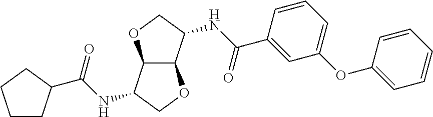

Prepared as in example 1 from cyclopentanecarboxylic acid and N-((3S,3aR,6S,6aR)-6-aminohexahydrofuro[3,2-b]furan-3-yl)-3-phenoxybenzamide (Example 2a) to yield N-((3S,3aR,6S,6aR)-6-(cyclopentanecarboxamido) hexahydrofuro[3,2-b]furan-3-yl)-3-phenoxybenzamide (4.4 mg, 40.4%). M+H=437.

### Example 16

### N-((3S,3aR,6S,6aR)-6-(Cyclopropanecarboxamido)hexahydrofuro[3,2-b]furan-3-yl)-3-methoxybenzamide

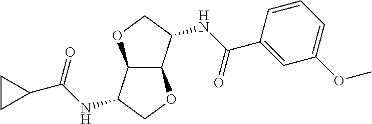

Prepared as in example 1 from 3-methoxybenzoic acid and N-((3S,3aR,6S,6aR)-6-aminohexahydrofuro[3,2-b]furan-3-yl)cyclopropanecarboxamide (Example 1a) to yield N-((3S,3aR,6S,6aR)-6-(cyclopropanecarboxamido)hexahydrofuro[3,2-h]furan-3-yl)-3-methoxybenzamide (4.9 mg, 56.5%). M+H=347.

### Example 17

### N-((3S,3aR,6S,6aR)-6-(3-Methylbutanamido)hexahydrofuro[3,2-b]furan-3-yl)-3-phenoxybenzamide

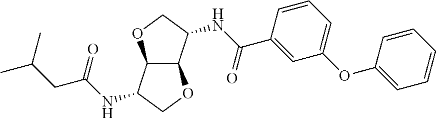

Prepared as in example 1 from 3-methylbutanoic acid and N-((3S,3aR,6S,6aR)-6-aminohexahydrofuro[3,2-b]furan-3-yl)-3-phenoxybenzamide (Example 2a) to yield N-((3S,3aR,6S,6aR)-6-(3-methylbutanamido)hexahydrofuro[3,2-b]furan-3-yl)-3-phenoxybenzamide (4.4 mg, 41.4%). M+H=425.

### Example 18

### 2-Methyl-N-((3S,3aR,6S,6aR)-6-(3-phenoxybenzamido) hexahydrofuro[3,2-b]furan-3-yl)benzamide

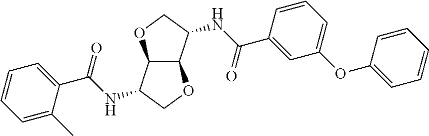

Prepared as in example 1 from 2-methylbenzoic acid and N-((3S,3aR,6S,6aR)-6-aminohexahydrofuro[3,2-b]furan-3-yl)-3-phenoxybenzamide (Example 2a) to yield 2-methyl-N-((3S,3aR,6S,6aR)-6-(3-phenoxybenzamido) hexahydrofuro[3,2-b]furan-3-yl)benzamide (4.7 mg, 41.0%). M+H=459.

### Example 19

### 2-Hydroxy-N-((3S,3aR,6S,6aR)-6-(3-phenoxybenzamido) hexahydrofuro[3,2-b]furan-3-yl)benzamide

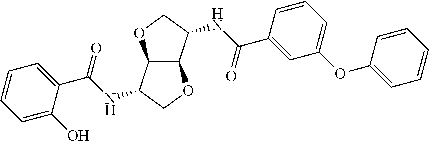

Prepared as in example 1 from 2-hydroxybenzoic acid and N-((3S,3aR,6S,6aR)-6-aminohexahydrofuro[3,2-b]furan-3-yl)-3-phenoxybenzamide (Example 2a) to yield 2-hydroxy-N-((3S,3aR,6S,6aR)-6-(3-phenoxybenzamido) hexahydrofuro[3,2-b]furan-3-yl)benzamide (4.1 mg, 35.7%). M+H=461.

### Example 20

### N-((3S,3aR,6S,6aR)-6-(4-Methylbenzamido)hexahydrofuro[3,2-b]furan-3-yl)-3-phenoxybenzamide

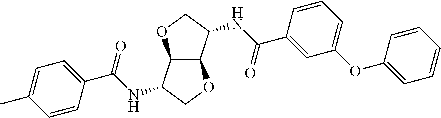

Prepared as in example 1 from 4-methylbenzoic acid and N-((3S,3aR,6S,6aR)-6-aminohexahydrofuro[3,2-b]furan-3-yl)-3-phenoxybenzamide (Example 2a) to yield N-((3S,3aR,6S,6aR)-6-(4-methylbenzamido)hexahydrofuro[3,2-b]furan-3-yl)-3-phenoxybenzamide (4.8 mg, 41.9%). M+H=459.

### Example 21

### N-((3S,3aR,6S,6aR)-6-(Cyclohexanecarboxamido)hexahydrofuro[3,2-b]furan-3-yl)-3-phenoxybenzamide

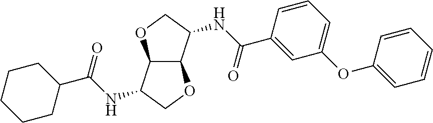

Prepared as in example 1 from cyclohexane carboxylic acid and N-((3S,3aR,6S,6aR)-6-aminohexahydrofuro[3,2-b]furan-3-yl)-3-phenoxybenzamide (Example 2a) to yield N-((3S,3aR,6S,6aR)-6-(cyclohexanecarboxamido) hexahydrofuro[3,2-b]furan-3-yl)-3-phenoxybenzamide (4.4 mg, 39.1%). M+H=451.

### Example 22

### N-((3S,3aR,6S,6aR)-6-Isobutyramidohexahydrofuro[3,2-b]furan-3-yl)-3-phenoxybenzamide

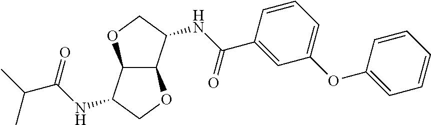

Prepared as in example 1 from isobutyric acid and N-((3S,3aR,6S,6aR)-6-aminohexahydrofuro[3,2-b]furan-3-yl)-3-phenoxybenzamide (Example 2a) to yield N-((3S,3aR,6S,6aR)-6-isobutyramidohexahydrofuro[3,2-b]furan-3-yl)-3-phenoxybenzamide (4.0 mg, 39.0%). M+H=411.

### Example 23

### N-((3S,3aR,6S,6aR)-6-(3-Phenoxybenzamido)hexahydrofuro[3,2-b]furan-3-yl)isonicotinamide

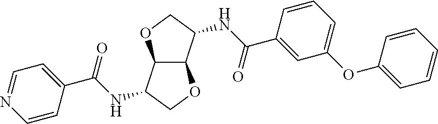

Prepared as in example 1 from isonicotinic acid and N-((3S,3aR,6S,6aR)-6-aminohexahydrofuro[3,2-b]furan-3-yl)-3-phenoxybenzamide (Example 2a) to yield N-((3S,3aR,6S,6aR)-6-(3-phenoxybenzamido)hexahydrofuro[3,2-b]furan-3-yl)isonicotinamide (4.9 mg, 44.0%). M+H=446.

### Example 24

### 3-(Cyclopentylmethoxy)-N-((3S,3aR,6S,6aR)-6-(cyclopropanecaboxamido)hexahydrofuro[3,2-b]furan-3-yl)benzamide

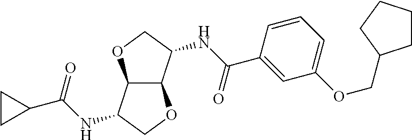

Prepared as in example 1 from 3-(cyclopentylmethoxy)benzoic acid (Example 24a) and N-((3S,3aR,6S,6aR)-6-aminohexahydrofuro[3,2-b]furan-3-yl)cyclopropanecarboxamide (Example 1a) to yield 3-(cyclopentyloxy)-N-((3S,3aR,6S,6aR)-6-(cyclopropanecaboxamido)hexahydrofuro[3,2-b]furan-3-yl)benzamide (4.1 mg, 39.4%). M+H=415.

### Example 24a

### 3-(Cyclopentylmethoxy)benzoic acid

Prepared as in example if from ethyl 3-(cyclopentylmethoxy)benzoate (Example 24b) to yield 3-(cyclopentylmethoxy)benzoic acid.

### Example 24b

### Ethyl 3-(cyclopentyloxy)benzoate

Prepared as in example 1g from ethyl 3-hydroxybenzoate and (bromomethyl)cyclopentane to yield ethyl 3-(cyclopentylmethoxy)benzoate.

### Example 25

### 3-Cyclobutoxy-N-((3S,3aR,6S,6aR)-6-(cyclopropanecaboxamido)hexahydrofuro[3,2-b]furan-3-yl)benzamide

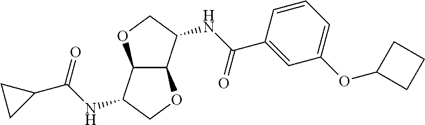

Prepared as in example 1 from 3-(cyclobutoxy)benzoic acid (Example 25a) and N-((3S,3aR,6S,6aR)-6-aminohexahydrofuro[3,2-b]furan-3-yl)cyclopropanecarboxamide (Example 1a) to yield 3-cyclobutoxy-N-((3S,3aR,6S,6aR)-6-(cyclopropanecaboxamido)hexahydrofuro[3,2-b]furan-3-yl)benzamide (3.9 mg, 40.2%). M+H=387.

### Example 25a

### 3-(Cyclobutoxy)benzoic acid

Prepared as in example if from ethyl 3-(cyclobutoxy)benzoate (Example 25b) to yield 3-(cyclobutoxy)benzoic acid.

### Example 25b

### Ethyl 3-(cyclobutoxy)benzoate

Prepared as in example 1g from ethyl 3-hydroxybenzoate and bromocyclobutane to yield ethyl 3-(cyclobutoxy)benzoate.

### Example 26

### 3-(Cyclohexyloxy)-N-((3S,3aR,6S,6aR)-6-(cyclopropanecaboxamido)hexahydrofuro[3,2-b]furan-3-yl)benzamide

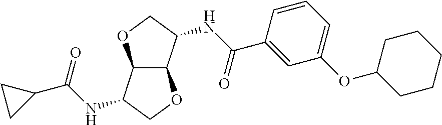

Prepared as in example 1 from 3-(cyclohexyloxy)benzoic acid (Example 26a) and N-((3S,3aR,6S,6aR)-6-aminohexahydrofuro[3,2-b]furan-3-yl)cyclopropanecarboxamide (Example 1a) to yield 3-(cyclohexyloxy)-N-((3S,3aR,6S,6aR)-6-(cyclopropanecaboxamido)hexahydrofuro[3,2-b]furan-3-yl)benzamide (4.1 mg, 39.4%). M+H=415.

### Example 26a

### 3-(Cyclohexyloxy)benzoic acid

Prepared as in example if from ethyl 3-(cyclohexyloxy)benzoate (Example 26b) to yield 3-(cyclohexyloxy)benzoic acid.

### Example 26b

### Ethyl 3-(cyclohexyloxy)benzoate

Prepared as in example 1g from ethyl 3-hydroxybenzoate and bromocyclohexane to yield ethyl 3-(cyclohexyloxy)benzoate.

### Example 27

### 3-(Cyclohexylmethoxy)-N-((3S,3aR,6S,6aR)-6-(cyclopropanecaboxamido)hexahydrofuro[3,2-b]furan-3-yl)benzamide

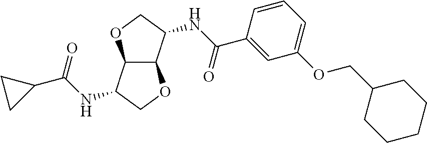

Prepared as in example 1 from 3-(cyclohexylmethoxy)benzoic acid (Example 27a) and N-((3S,3aR,6S,6aR)-6-aminohexahydrofuro[3,2-b]furan-3-yl)cyclopropanecarboxamide (Example 1a) to yield 3-(cyclohexylmethoxy)-N—R3S,3aR,6S,6aR)-6-(cyclopropanecaboxamido)hexahydrofuro[3,2-b]furan-3-yl)benzamide (3.7 mg, 34.6%). M+H=429.

### Example 27a

### 3-(Cyclohexylmethoxy)benzoic acid

Prepared as in example if from ethyl 3-(cyclohexylmethoxy)benzoate (Example 27b) to yield 3-(cyclohexylmethoxy)benzoic acid.

### Example 27b

### Ethyl 3-(cyclohexylmethoxy)benzoate

Prepared as in example 1g from ethyl 3-hydroxybenzoate and (bromomethyl)cyclohexane to yield ethyl 3-(cyclohexylmethoxy)benzoate.

### Example 28

### 3-(Cycloheptyloxy)-N-((3S,3aR,6S,6aR)-6-(cyclopropanecaboxamido)hexahydrofuro[3,2-b]furan-3-yl)benzamide

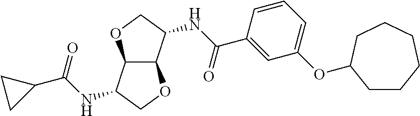

Prepared as in example 1 from 3-(cycloheptyloxy)benzoic acid (Example 28a) and N-((3S,3aR,6S,6aR)-6-aminohexahydrofuro[3,2-b]furan-3-yl)cyclopropanecarboxamide (Example 1a) to yield 3-(cycloheptyloxy)-N-((3S,3aR,6S,6aR)-6-(cyclopropanecaboxamido)hexahydrofuro[3,2-b]furan-3-yl)benzamide (4.5 mg, 42.1%). M+H=429.

### Example 28a

### 3-(Cycloheptyloxy)benzoic acid

Prepared as in example if from ethyl 3-(cycloheptyloxy)benzoate (Example 28b) to yield 3-(cycloheptyloxy)benzoic acid.

### Example 28b

### Ethyl 3-(cycloheptyloxy)benzoate

Prepared as in example 1g from ethyl 3-hydroxybenzoate and bromocycloheptane to yield ethyl 3-(cycloheptyloxy)benzoate.

### Example 29

### N-((3S,3aR,6S,6aR)-6-(Cyclopropanecaboxamido)hexahydrofuro[3,2b]furan-3-yl)-3-(pentan-3-yloxy)benzamide

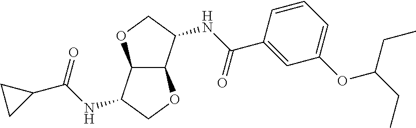

Prepared as in example 1 from 3-(pentan-3-yloxy)benzoic acid (Example 29a) and N-((3S,3aR,6S,6aR)-6-aminohexahydrofuro[3,2-b]furan-3-yl)cyclopropanecarboxamide (Example 1a) to yield N-((3S,3aR,6S,6aR)-6-(cyclopropanecaboxamido)hexahydrofuro[3,2-b]furan-3-yl)-3-(pentan-3-yloxy)benzamide (3.8 mg, 37.7%). M+H=403.

### Example 29a

### 3-(Pentan-3-yloxy)benzoic acid

Prepared as in example if from ethyl 3-(pentan-3-yloxy)benzoate (Example 29b) to yield 3-(pentan-3-yloxy)benzoic acid.

### Example 29b

### Ethyl 3-(pentan-3-yloxy)benzoate

Prepared as in example 1g from ethyl 3-hydroxybenzoate and 3-bromopentane to yield ethyl 3-(pentan-3-yloxy)benzoate.

### Example 30

### 3-sec-Butoxy-N-((3S,3aR,6S,6aR)-6-(cyclopropanecaboxamido)hexahydrofuro[3,2-b]furan-3-yl)benzamide

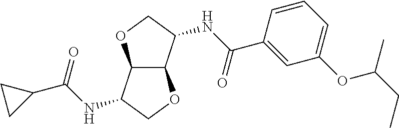

Prepared as in example 1 from 3-sec-butoxybenzoic acid (Example 30a) and N-((3S,3aR,6S,6aR)-6-aminohexahydrofuro[3,2-b]furan-3-yl)cyclopropanecarboxamide (Example 1a) to yield 3-sec-butoxy-N-((3S,3aR,6S,6aR)-6-(cyclopropanecaboxamido)hexahydrofuro[3,2-b]furan-3-yl)benzamide (3.5 mg, 36.0%). M+H=389.

### Example 30a

### 3-sec-Butoxybenzoic acid

Prepared as in example if from ethyl 3-sec-butoxybenzoate (Example 30b) to yield 3-sec-butoxybenzoic acid.

### Example 30b

### Ethyl 3-sec-butoxybenzoate

Prepared as in example 1g from ethyl 3-hydroxybenzoate and 2-bromobutane to yield ethyl 3-sec-butoxybenzoate.

### Example 31

### N-((3S,3aR,6S,6aR)-6-(Cyclopropanecaboxamido)hexahydrofuro[3,2-b]furan-3-yl)-3-isobutoxybenzamide

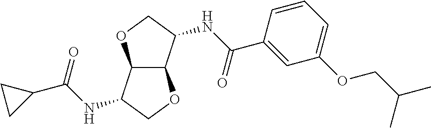

Prepared as in example 1 from 3-isobutoxybenzoic acid (Example 31a) and N-((3S,3aR,6S,6aR)-6-aminohexahydrofuro[3,2-b]furan-3-yl)cyclopropanecarboxamide (Example 1a) to yield N-((3S,3aR,6S,6aR)-6-(cyclopropanecaboxamido)hexahydrofuro[3,2-b]furan-3-yl)-3-isobutoxybenzamide (3.5 mg, 36.1%). M+H=389.

### Example 31a

### 3-Isobutoxybenzoic acid

Prepared as in example 1f from ethyl 3-isobutoxybenzoate (Example 31b) to yield 3-isobutoxybenzoic acid.

### Example 31b

### Ethyl 3-isobutoxybenzoate

Prepared as in example 1g from ethyl 3-hydroxybenzoate and 1-bromo-2-methylpropane to yield ethyl 3-isobutoxybenzoate.

All publications and patent applications herein are incorporated by reference to the same extent as if each individual publication or patent application was specifically and individually indicated to be incorporated by reference.

The foregoing detailed description has been given for clearness of understanding only and no unnecessary limitations should be understood therefrom as modifications will be obvious to those skilled in the art. It is not an admission that any of the information provided herein is prior art or relevant to the presently claimed inventions, or that any publication specifically or implicitly referenced is prior art.

Embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

We claim:

1. A compound having structural formula (I): 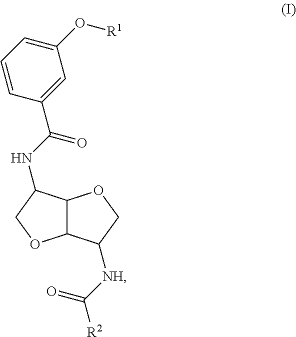
or the salt, solvate, ester, or N-oxide thereof,
wherein:
R¹ is alkyl, substituted alkyl, alkenyl, substituted alkenyl, aryl, substituted aryl, carbocyclyl, or substituted carbocyclyl;
R² is alkyl, substituted alkyl, alkenyl, substituted alkenyl, aryl, substituted aryl, carbocyclyl, substituted carbocyclyl, heteroaryl, or substituted heteroaryl; and
with the proviso that when R² is cyclopropyl; then R¹ is not phenyl, tolyl, methoxyphenyl, or pyridyl.

2. The compound of claim 1, wherein
R¹ is alkyl, substituted alkyl, cycloalkyl, substituted cycloalkyl, phenyl, substituted phenyl; and
R² is alkyl, substituted alkyl, cycloalkyl, substituted cycloalkyl, phenyl, substituted phenyl, pyridyl, or substituted pyridyl.

3. The compound of claim 1, having structural formula (Ia): 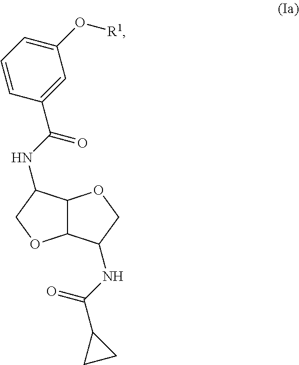
wherein:
R¹ is alkyl, substituted alkyl, cycloalkyl, substituted cycloalkyl, or substituted phenyl.

4. The compound of claim 1, having structural formula (Ib): 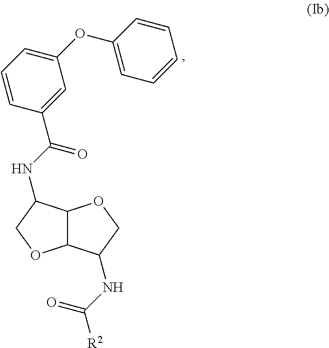
wherein:
R² is alkyl, substituted alkyl, cycloalkyl, substituted cycloalkyl, phenyl, substituted phenyl, pyridyl, or substituted pyridyl.

5. The compound of claim 1, having a structural formula selected from the group consisting of

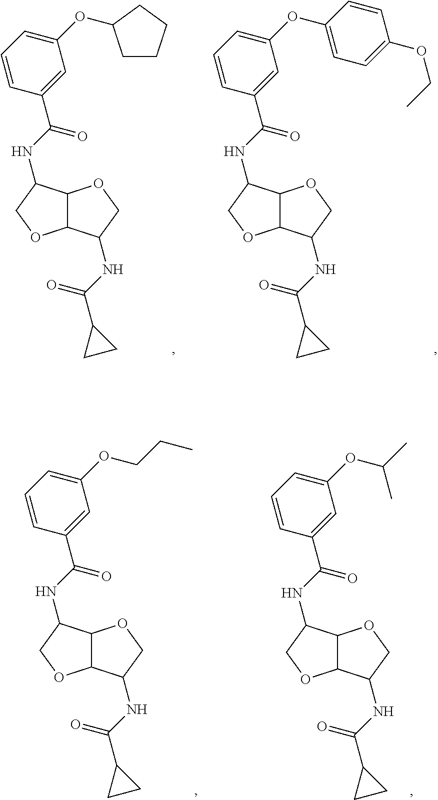

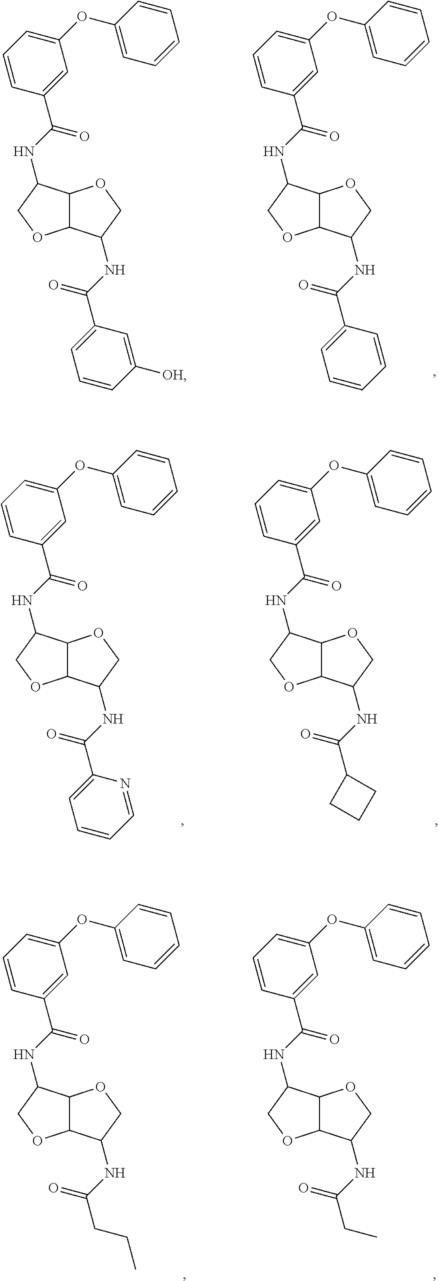

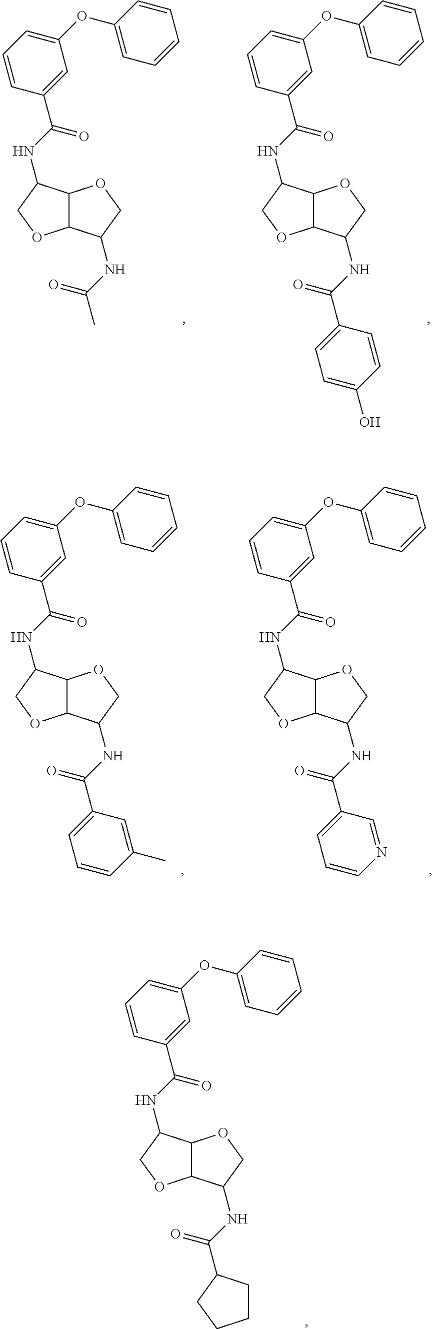

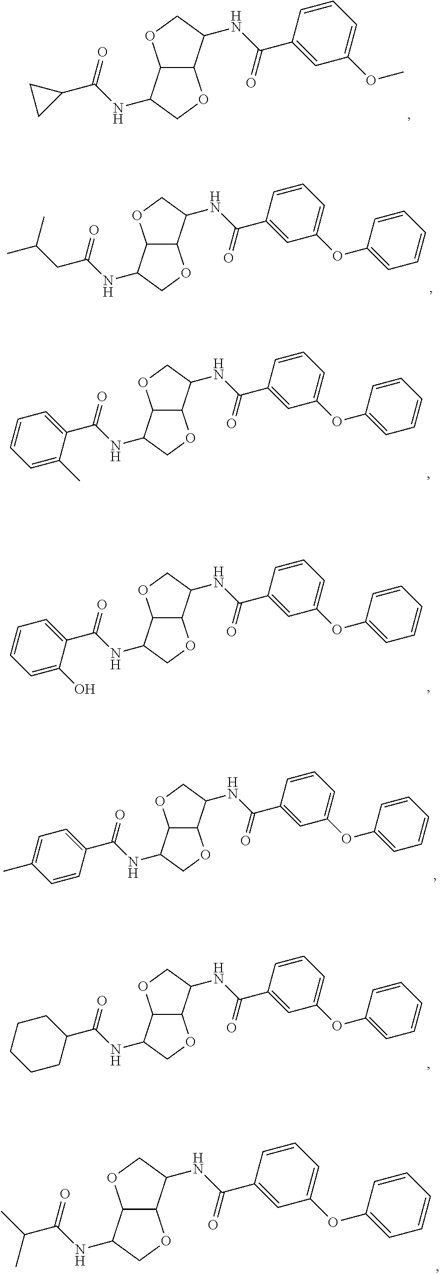

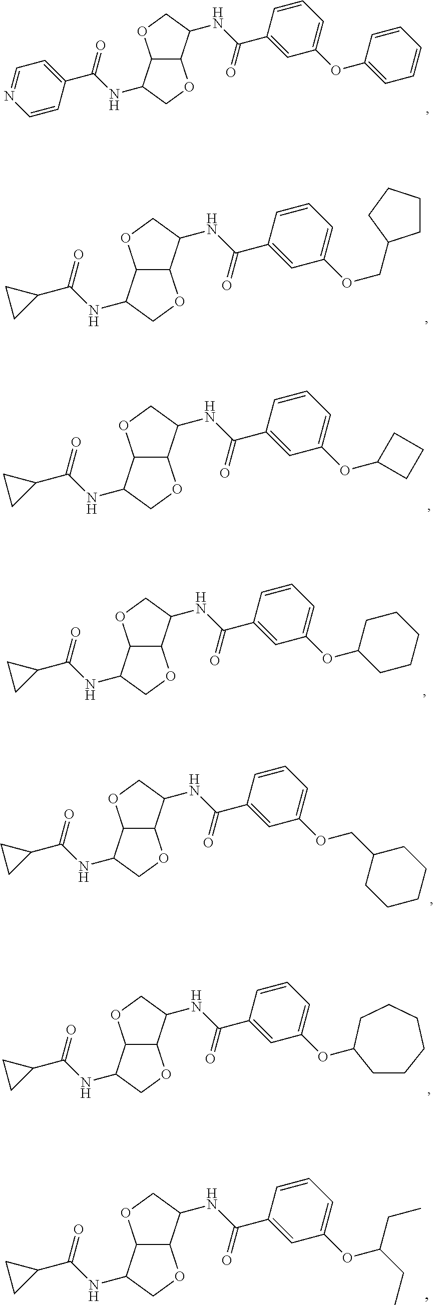

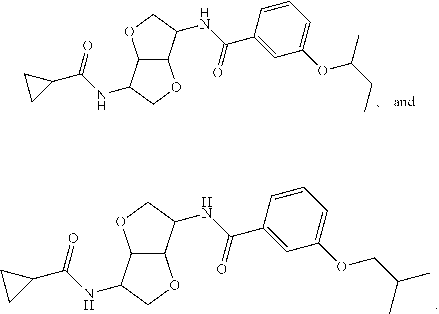

6. A composition comprising a compound having structural formula (I): 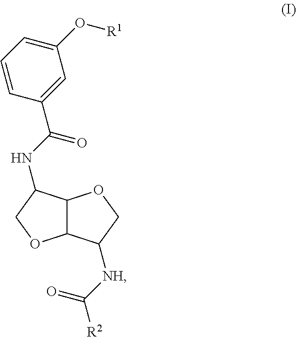
or the salt, solvate, ester, or N-oxide thereof,
wherein:
R¹ is alkyl, substituted alkyl, alkenyl, substituted alkenyl, aryl, substituted aryl, carbocyclyl, or substituted carbocyclyl;
R² is alkyl, substituted alkyl, alkenyl, substituted alkenyl, aryl, substituted aryl, carbocyclyl, substituted carbocyclyl, heteroaryl, or substituted heteroaryl; and
with the proviso that when R² is cyclopropyl; then R¹ is not phenyl, tolyl, methoxyphenyl, or pyridyl.

7. The composition of claim 6, which comprises an ingestible composition.

8. The composition of claim 7, which comprises a food or beverage.

9. A method for modulating the savory taste of a composition comprising combining the composition with at least one compound of formula (I): 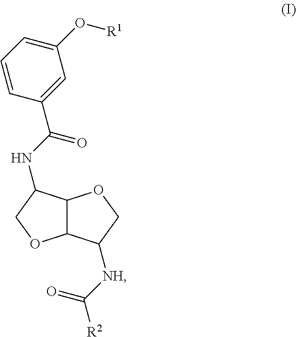
or the salt, solvate, ester, or N-oxide thereof,
wherein:
R¹ is alkyl, substituted alkyl, alkenyl, substituted alkenyl, aryl, substituted aryl, carbocyclyl, or substituted carbocyclyl;
R² is alkyl, substituted alkyl, alkenyl, substituted alkenyl, aryl, substituted aryl, carbocyclyl, substituted carbocyclyl, heteroaryl, or substituted heteroaryl; and
with the proviso that when R² is cyclopropyl; then R¹ is not phenyl, tolyl, methoxyphenyl, or pyridyl.

10. A method of preparing a compound of formula (II) in a solid form substantially without chromatographic purification: 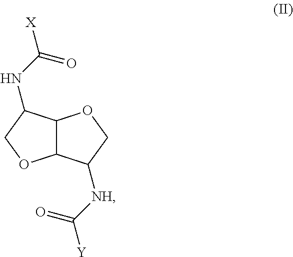
comprising
converting isomannide to a compound of formula (IIa): 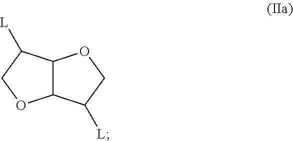
reacting the compound of formula (IIa) with phenylmethanamine to obtain a compound of formula (IIb): 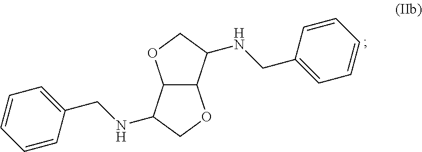
reducing the compound of formula (IIb) to a compound of formula (IIc): 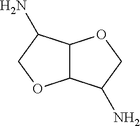
and
reacting the compound of formula (IIc) sequentially with a compound of formula (IId): 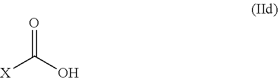
and a compound of formula (IIe): 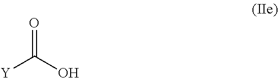
in the presence of one or more activating reagent;
wherein
L is a leaving group; and
X and Y are each independently alkyl, substituted alkyl, alkenyl, substituted alkenyl, alkynyl, substituted alkylnyl, alkoxy, alkylamine, heteroalkyl, substituted heteroalkyl, aryl, substituted aryl, arylalkyl, substituted arylalkyl, arylalkenyl, substituted arylalkenyl, heteroaryl, substituted heteroaryl, heterocyclyl, substituted heterocyclyl, heteroarylalkyl, substituted heteroarylalkyl, heterocyclylalkyl, substituted heterocyclylalkyl, carbocyclyl, or substituted carbocyclyl.

11. The method of claim 10, wherein L is halo, —OMs, —OTs, or —OTf.

12. The method of claim 10, wherein
X and Y are each independently selected from alkyl, substituted alkyl, alkenyl, substituted alkenyl, alkylamine, aryl, substituted aryl, heteroaryl, substituted heteroaryl, arylalkyl, substituted arylalkyl, heterocyclyl, substituted heterocyclyl, heteroarylalkyl, substituted heteroarylalkyl, carbocyclyl, or substituted carbocyclyl.

13. The method of claim 10, wherein the one or more activating reagent is selected from a group consisting of a carbodiimide, a benzotrizaole compound, a phosphorus oxide or chloride, and a combination thereof.

14. The method of claim 10, wherein the reduction of the compound of formula (IIb) is carried out by catalytic hydrogenation.
